(19) United States
(12) Patent Application Publication
(10) Pub. No.: US 2021/0260078 A1
(43) Pub. Date: Aug. 26, 2021

(54) LOW DOSAGE INTRANASAL AMINOSTEROL DOSAGE FORMS AND METHODS OF USING THE SAME

(71) Applicant: Enterin, Inc., Philadelphia, PA (US)

(72) Inventors: Michael Zasloff, Philadelphia, PA (US); Denise Barbut, Philadelphia, PA (US)

(73) Assignee: Philadelphia, Philadelphia, PA (US)

(21) Appl. No.: 17/264,284

(22) PCT Filed: Aug. 2, 2019
(86) PCT No.: PCT/US2019/044882

§ 371 Date: Jan. 28, 2021

Related U.S. Application Data
(60) Provisional application No. 62/714,470, filed on Aug. 3, 2018.

(51) Int. Cl. A61K 31/575; A61K 9/00; A61K 47/10; A61K 47/26

(52) U.S. Cl. CPC A61K 31/575; A61K 47/26; A61K 47/10; A61K 9/0043

(57) ABSTRACT

This invention relates to novel, effective methods and compositions for mucosal, especially intranasal, delivery of a low dosage of an aminosterol for treatment and prevention of certain afflictions. Any disease or condition amenable to treatment with an aminosterol can be treated using the intranasal low dose aminosterol compositions of the invention.

### CROSS-REFERENCE TO RELATED APPLICATION

This application claims the priority benefits under 35 USC § 119 to U.S. provisional Application No. 62/714,470, filed Aug. 3, 2018, the entire contents of which is incorporated herein by reference in its entirety.

### FIELD OF THE INVENTION

This invention relates to low dosage intranasal compositions of aminosterols and methods of treatment utilizing the same.

### BACKGROUND OF THE INVENTION

Aminosterols are amino derivatives of a sterol. Examples of aminosterols include squalamine and Aminosterol 1436 (also known as trodusquemine and MSI-1436).

Squalamine is a unique compound with a structure of a bile acid coupled to a polyamine (spermidine):

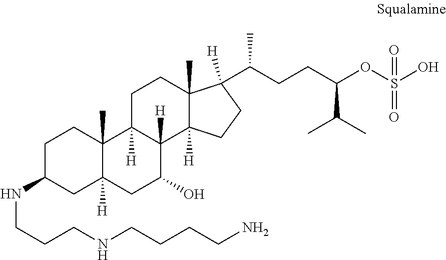

The discovery of squalamine, the structure of which is shown above, was reported by Michael Zasloff in 1993 (U.S. Pat. No. 5,192,756). Squalamine was discovered in various tissues of the dogfish shark (Squalus acanthias) in a search for antibacterial agents. The most abundant source of squalamine is in the livers of Squalus acanthias, although it is found in other sources, such as lampreys (Yun et al., 2007).

Several clinical trials have been conducted relating to the use of squalamine, including the following:

(1) ClinicalTrials.gov Identifier NCT01769183 for “Squalamine for the Treatment in Proliferative Diabetic Retinopathy,” by Elman Retina Group (6 participants; study completed August 2014);

(2) ClinicalTrials.gov Identifier NCT02727881 for “Efficacy and Safety Study of Squalamine Ophthalmic Solution in Subjects With Neovascular AMD (MAKO),” by Ohr Pharmaceutical Inc. (230 participants; study completed December 2017);

(3) ClinicalTrials.gov Identifier NCT02614937 for “Study of Squalamine Lactate for the Treatment of Macular Edema Related to Retinal Vein Occlusion,” by Ohr Pharmaceutical Inc. (20 participants; study completed December 2014);

(4) ClinicalTrials.gov Identifier NCT01678963 for “Efficacy and Safety of Squalamine Lactate Eye Drops in Subjects With Neovascular (Wet) Age-related Macular Degeneration (AMD),” by Ohr Pharmaceutical Inc. (142 participants; study completed March 2015);

(5) ClinicalTrials.gov Identifier NCT00333476 for “A Study of MSI-1256F (Squalamine Lactate) To Treat “Wet” Age-Related Macular Degeneration,” by Genaera Corporation (140 participants; study terminated);

(6) ClinicalTrials.gov Identifier NCT00094120 for “MSI-1256F (Squalamine Lactate) in Combination With Verteporfin in Patients With “Wet” Age-Related Macular Degeneration (AMD),” by Genaera Corporation (60 participants; study completed February 2007); and

(7) ClinicalTrials.gov Identifier NCT00089830 for “A Safety and Efficacy Study of MSI-1256F (Squalamine Lactate) To Treat “Wet” Age-Related Macular Degeneration,” by Genaera Corporation (120 participants; study completed May 2007).

Aminosterol 1436 is an aminosterol isolated from the dogfish shark, which is structurally related to squalamine (U.S. Pat. No. 5,840,936; Rao, Shinnar et al. 2000). It is also known as MSI-1436, trodusquemine and produlestan.

Several clinical trials have been conducted relating to the use of Aminosterol 1436:

(1) ClinicalTrials.gov Identifier NCT00509132 for “A Phase I, Double-Blind, Randomized, Placebo-Controlled Ascending IV Single-Dose Tolerance and Pharmacokinetic Study of Trodusquemine in Healthy Volunteers,” by Genaera Corp.;

(2) ClinicalTrials.gov Identifier NCT00606112 for “A Single Dose, Tolerance and Pharmacokinetic Study in Obese or Overweight Type 2 Diabetic Volunteer,” by Genaera Corp.;

(3) ClinicalTrials.gov Identifier NCT00806338 for “An Ascending Multi-Dose, Tolerance and Pharmacokinetic Study in Obese or Overweight Type 2 Diabetic Volunteers,” by Genaera Corp.; and

(4) ClinicalTrials.gov Identifier: NCT02524951 for “Safety and Tolerability of MSI-1436C in Metastatic Breast Cancer,” by DepyMed Inc.

There is a need for new dosage forms of aminosterols which provide benefits not seen with current dosage forms. The present invention satisfies this need.

### SUMMARY OF THE INVENTION

The present invention is directed to low dose, intranasal dosage forms of aminosterols. In one embodiment, encompassed is a pharmaceutical composition formulated for intranasal administration, comprising a low dosage of at least one aminosterol or a pharmaceutically acceptable salt or derivative thereof, wherein the dosage of the aminosterol does not result in a pharmacological effect when given orally or by injection.

The low dosage of the aminosterol can be, for example, between about 0.001 to about 6 mg. In another embodiment, the low dosage of the aminosterol can be, for example, about 0.001 to 4 mg/kg. In another embodiment, the low dosage of an aminosterol is a dosage which is subtherapeutic when given orally or by injection.

The pharmaceutical compositions of the invention preferably comprise a pharmaceutically acceptable grade of at least one aminosterol or a pharmaceutically acceptable salt or derivative thereof. The aminosterol can be, for example: (a) isolated from the liver of Squalus acanthias; (b) a squalamine isomer; (c) squalamine; (d) the phosphate salt of squalamine; (e) comprises a sterol nucleus and a polyamine attached at any position on the sterol, such that the molecule exhibits a net charge of at least +1 (wherein the polyamine may contribute to the net charge); (f) comprises a bile acid nucleus and a polyamine, attached at any position on the bile acid, such that the molecule exhibits a net charge of at least +1 (wherein the polyamine may contribute to the net charge); (g) a derivative modified to include one or more of the following: (i) substitutions of the sulfate by a sulfonate, phosphate, carboxylate, or other anionic moiety chosen to circumvent metabolic removal of the sulfate moiety and oxidation of the cholesterol side chain; (ii) replacement of a hydroxyl group by a non-metabolizable polar substituent, such as a fluorine atom, to prevent its metabolic oxidation or conjugation; and (iii) substitution of one or more ring hydrogen atoms to prevent oxidative or reductive metabolism of the steroid ring system; (h) a derivative of squalamine modified through medical chemistry to improve bio-distribution, ease of administration, metabolic stability, or any combination thereof, (i) aminosterol 1436; (j) an isomer of aminosterol 1436; (k) the phosphate salt of aminosterol 1436; (l) a pharmaceutically acceptable salt of the aminosterol, wherein the salt has low mucosal irritation; (m) a synthetic aminosterol; and/or (n) a free base of the aminosterol.

The pharmaceutical compositions may further comprise one or more of an aqueous carrier, a buffer, a sugar; and/or a polyol compound. For example, the sugar can be lactose and the polyol compound can be glycerin. In another embodiment, the composition can comprise an aqueous carrier and glycerin at about a 2:1 ratio.

Also encompassed are methods of treatment comprising administering the low dosage aminosterol intranasal compositions of the invention to a subject in need. The subject to be treated can be a human, such as an infant, toddler, school-aged child, teenager, young adult, adult, or elderly subject.

The methods of the invention encompass combination treatment, where the intransally administered aminosterol is administered in combination with at least one additional active agent to achieve either an additive or synergistic effect. In one embodiment, the additional active agent is an aminosterol which is delivered orally. For example, the aminosterol administered intranasally can be aminosterol 1436 or a salt or derivative thereof, and the aminosterol administered orally can be squalamine or a salt or derivative thereof. The additional active agent the additional active agent can be administered via a method such as concomitantly, as an admixture, separately and simultaneously or concurrently, or separately and sequentially.

In one embodiment, the invention encompasses methods of treating a subject at risk for developing, or is suffering from, neurodegeneration, and the method results in treating, preventing, and/or delaying the progression and/or onset of neurodegeneration in the subject. In this method, the neurodegeneration can be age-related, correlated with age-related dementia, correlated with a neurodisease, and/or the neurodegeneration can be correlated with one or more conditions or diseases selected from the group consisting of Alzheimer's disease, Parkinson's disease, Lewy Body dementia, frontotemporal dementia, supranuclear palsy, multi-system atrophy, Parkinsonism, amyotrophic lateral sclerosis (ALS), Huntington's Disease, schizophrenia, Friedreich's ataxia, Multiple sclerosis (MS), spinal muscular atrophy, progressive nuclear palsy, degenerative processes associated with aging, dementia of aging, Guadeloupian Parkinsonism, spinocerebellar ataxia, or vascular dementia.

In one embodiment, wherein progression or onset of the neurodegeneration is slowed, halted, or reversed over a defined time period following administration of the pharmaceutical composition, as measured by a medically-recognized technique. In another embodiment, the neurodegeneration is positively impacted by administration of the pharmaceutical composition. In yet another embodiment, the positive impact and/or progression of neurodegeneration is measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis. In a further embodiment, the progression or onset of neurodegeneration is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In another embodiment of the invention, encompassed are methods of treatment where the subject is at risk of developing, or suffers from, a sleep disorder or sleep disturbance. In this embodiment, administration of the composition can decrease the occurrence of at least one symptom of the sleep disorder or disturbance.

The sleep disorder can comprise a loss of diurnal rhythm (Circadian rhythm). The loss of diurnal rhythm can be caused by, for example, dysfunction of the suprachiasmatic nucleus, and administration of the aminosterol composition of the invention reverses the dysfunction of the suprachiasmatic nucleus, restores the diurnal rhythm, and treats the sleep disorder. In another embodiment, the loss of diurnal rhythm is caused by dysfunction of the enteric nervous system, and administration of the aminosterol composition of the invention reverses the dysfunction of the enteric nervous system, restores the diurnal rhythm, and treats the sleep disorder. In another embodiment, the loss of diurnal rhythm is caused by dysfunction of the olfactory nervous system, and administration of the aminosterol composition of the invention reverses the dysfunction of olfactory system, restores the diurnal rhythm, and treats the sleep disorder. In another embodiment, the loss of diurnal rhythm is caused by visual loss, and administration of the aminosterol composition of the invention reverses dysfunction of the circadian rhythm caused by visual loss. In another embodiment, the loss of diurnal rhythm is caused by jet lag, and administration of the aminosterol composition of the invention reverses dysfunction of the circadian rhythm caused by jet lag. Finally, in another embodiment, the loss of diurnal rhythm is caused by night-shift work, and administration of the aminosterol composition of the invention reverses dysfunction of the circadian rhythm caused by night-shift work.

In one embodiment, the sleep disorder comprises a delay in sleep onset, sleep fragmentation, REM-behavior disorder, sleep-disordered breathing including snoring and apnea, day-time sleepiness, micro-sleep episodes, narcolepsy, hallucinations, or any combination thereof. In another embodiment, the REM-behavior disorder comprises vivid dreams, nightmares, and acting out the dreams by speaking or screaming, or fidgeting or thrashing of arms or legs during sleep.

In a further embodiment, the sleep disorder is associated with a neurodegenerative disorder. In an exemplary embodiment, treating the sleep disorder prevents or delays the onset or progression of a neurodegenerative disorder. The neurodegenerative disorder can be, for example, selected from the group consisting of Alzheimer's disease, Parkinson's disease, Lewy Body dementia, frontotemporal dementia, supranuclear palsy, multi-system atrophy, Parkinsonism, amyotrophic lateral sclerosis (ALS), Huntington's Disease, schizophrenia, Friedreich's ataxia, Multiple sclerosis (MS), spinal muscular atrophy, progressive nuclear palsy, degenerative processes associated with aging, dementia of aging, Guadeloupian Parkinsonism, spinocerebellar ataxia, and vascular dementia.

In one embodiment, the method of the invention results in a positive change in the sleeping pattern of the subject. For example, the positive change can be defined as: (a) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (b) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In another embodiment of the invention, as a result of the method the subject obtains the total number of hours of sleep recommended by a medical authority for the age group of the subject.

The invention also encompasses methods of treatment, wherein the subject suffers anosmia or from hyposmia, and the method result in either complete or partial restoration of the subject's sense of smell. In one embodiment, the method results in improving the subject's sense of smell by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In this method, the subject may have experienced head trauma, and/or the subject may be at risk of developing Parkinson's disease, and/or the subject may be at risk of developing a neurodisease.

In another embodiment of the invention, encompassed are methods of treatment where the subject suffers from, is or at risk of developing, hallucinations. The hallucination can comprise, for example, a visual, auditory, tactile, gustatory or olfactory hallucination. In another embodiment, the hallucination can be the result of a neurodegenerative disorder, a psychiatric disorder, a neurological disorder, a brain tumor, a sensory loss, and/or dysfunction of the enteric nervous system. In one embodiment, the sensory loss is visual, auditory, gustatory, tactile, or olfactory.

In one embodiment for this method, the neurodegenerative disorder is selected from the group consisting of Alzheimer's disease, Parkinson's disease, Lewy Body dementia, frontotemporal dementia, supranuclear palsy, multi-system atrophy, Parkinsonism, amyotrophic lateral sclerosis (ALS), Huntington's Disease, schizophrenia, Friedreich's ataxia, Multiple sclerosis (MS), spinal muscular atrophy, progressive nuclear palsy, degenerative processes associated with aging, dementia of aging, Guadeloupian Parkinsonism, spinocerebellar ataxia, and vascular dementia.

The neurodegenerative or neurological disorder can be the result of, for example, a sleep disorder, a focal brain lesion, a focal brain lesion which is occipital lobe lesions or temporal lobe lesions, a temporal lobe lesion selected from the group consisting of lesions of the uncinate gyrus, cerebral peduncles, and substantia nigra, a diffuse involvement of the cerebral cortex, a diffuse involvement of the cerebral cortex caused by a viral infectious disease, a diffuse involvement of the cerebral cortex caused by a viral infectious disease, wherein the viral infectious disease is selected from the group consisting of acute metabolic encephalopathies, encephalitis, and meningitis, a diffuse involvement of the cerebral cortex caused by a cerebral vasculitis condition, a diffuse involvement of the cerebral cortex caused by a cerebral vasculitis condition, wherein the cerebral vasculitis condition is caused by an autoimmune disorder, a bacterial or viral infection, or a systemic vasculitis, and/or a diffuse involvement of the cerebral cortex caused by a cerebral vasculitis condition, wherein the cerebral vasculitis condition is caused by an autoimmune disorder which is Systemic Lupus Erythematosus (SLE).

In another embodiment for this method, the psychiatric disorder is selected from the group consisting of Bipolar disorder, Borderline personality disorder, Depression (mixed), Dissociative identity disorder, Generalized anxiety disorder, Major depression, Obsessive compulsive disorder, Post-traumatic stress disorder, Psychosis (NOS), Schizoaffective disorder, and Schizophrenia.

In methods of treating a subject suffering from, is or at risk of developing, hallucinations, where the hallucination may be the result of a neurodegenerative disorder, administration of the aminosterol reverses the dysfunction caused by the neurodegenerative or neurological disorder and treats and/or prevents the hallucination. In another embodiment, where the hallucination may be the result of a psychiatric disorder, administration of the aminosterol reverses the dysfunction caused by the psychiatric disorder and treats and/or prevents the hallucination. In another embodiment, where the hallucination may be the result of a sensory loss, administration of the aminosterol reverses the dysfunction caused by the sensory loss and treats and/or prevents the hallucination. Finally, where the hallucination may be the result of dysfunction of the enteric nervous system, administration of the aminosterol reverses the dysfunction of the enterin nervous system and treats and/or prevents the hallucination.

In one embodiment, the method results in a decreased number or severity of hallucinations of the subject. For example, the decrease in number or severity in hallucinations can be defined as a reduction in occurrences or severity of hallucinations selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%. In another embodiment, the method of the invention results in the subject being hallucination-free.

Also encompassed are methods of treatment where the subject suffers from, is or at risk of developing, depression. In one embodiment, the method results in improvement in a subject's depression, as measured by one or more clinically-recognized depression rating scale. For example, the improvement can be in one or more depression characteristics selected from the group consisting of mood, behavior, bodily functions such as eating, sleeping, energy, and sexual activity, and/or episodes of sadness or apathy. In another embodiment, the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%.

In one embodiment, administration of the intranasal aminosterol composition triggers neurogenesis, which functions to combat depression.

Also encompassed are methods of treatment where the subject suffers from, is or at risk of developing, autism. In one embodiment, the method results in improvement in one or more of the subject's autism characteristics or behaviors, as measured by a clinically-recognized rating scale. For example, the method can result in improvement in one or more autism characteristics or behaviors selected from the group consisting of social skills, repetitive behaviors, speech, nonverbal communication, sensory sensitivity, behavior, social interaction, and communication skills, as measured using a clinically-recognized scale. In another embodiment, the improvement a subject experiences following treatment in one or more autism characteristics or behaviors is about 5, about 10, about 15, about 20, about 25, about 30, about 35, about 40, about 45, about 50, about 55, about 60, about 65, about 70, about 75, about 80, about 85, about 90, about 95 or about 100%.

In one embodiment, administration of the intranasal aminosterol composition triggers neurogenesis, which functions to combat one or more autism characteristics.

Also encompassed are methods of treatment where the subject suffers from, is or at risk of developing, schizophrenia. In one embodiment, the method results in improvement in one or more schizophrenia characteristics or behaviors, as measured using a clinically recognized rating scale. In another embodiment, the schizophrenia characteristics or behaviors are selected from the group consisting of unclear or confusing thinking, reduced social engagement, reduced emotional expression, abnormal social behavior, failure to understand reality, lack of motivation, and hearing voices that others do not hear, as measured using a clinically-recognized scale. In yet another embodiment, the improvement a subject experiences in one or more schizophrenia characteristics or behaviors following treatment is about 5, about 10, about 15, about 20, about 25, about 30, about 35, about 40, about 45, about 50, about 55, about 60, about 65, about 70, about 75, about 80, about 85, about 90, about 95 or about 100%.

In one embodiment, administration of the intranasal aminosterol composition triggers neurogenesis, which functions to combat one or more schizophrenia characteristics.

Also encompassed are methods of treatment where the subject suffers from, is or at risk of developing, an inflammatory disease or condition caused by excessive expression or concentration of alpha synuclein in the subject. In one embodiment, the method results in a decrease in intensity of inflammation, blood levels of inflammatory markers, inflammatory markers in tissue, number of inflammatory cells in tissue, or any combination thereof, as compared to a control or as compared to the qualitative or quantitative amount from the same patient or subject prior to treatment. In another embodiment, the method results in a decrease in concentration of alpha synuclein in the subject.

In yet another embodiment, the decrease in alpha-synuclein concentration in is measured qualitatively, quantitatively, or semi-quantitatively by one or more methods selected from the group consisting of: (a) first determining the concentration of alpha-synuclein in a tissue sample from the subject prior to treatment, followed by: (i) after treatment determining the alpha-synuclein concentration in the same tissue type from the same subject; or (ii) after treatment comparing the alpha-synuclein concentration in the same tissue type to a control; (b) measuring the intensity of inflammation over time; (c) measuring the amount of inflammatory markers over time; (d) measuring the amount of inflammatory markers in blood, plasma, or tissue over time, either qualitatively or quantitatively; (e) measuring the amount of one or more inflammatory marker cytokines in blood, plasma, or tissue over time, either qualitatively or quantitatively; (f) measuring the amount of one or more plasma markers of inflammation such as TNF, IL-8, or CRP in blood, plasma, or tissue over time, either qualitatively or quantitatively; and (g) measuring the amount of inflammatory cells in blood, plasma, or tissue over time, either qualitatively or quantitatively. In another embodiment, the decrease in alpha-synuclein concentration is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In yet another embodiment, the method is applied to a patient population susceptible to excessive expression of alpha-synuclein, resulting in an excessive or high concentration of alpha-synuclein.

Both the foregoing summary and the following description of the drawings and detailed description are exemplary and explanatory. They are intended to provide further details of the invention, but are not to be construed as limiting. Other objects, advantages, and novel features will be readily apparent to those skilled in the art from the following detailed description of the invention.

### DESCRIPTION OF THE FIGURES

FIG. 1: Shows the accumulation of Aminosterol 1436 within the centers of the brain that control growth, maturation, and senescence following intravenous administration to a rat via a peripheral vein via intravenous (IV) administration (FIGS. 1B and C), or injected via intracerebroventricular (ICV) administration directly into the 3^{rd} ventricle of the brain (FIG. 1A).

FIG. 2: FIG. 2A shows the in vivo distribution of the aminosterol Aminosterol 1436 administered intraperitoneal (IP) or ICV as compared to vehicle (administered IP) in the Arc (arcuate nucleus of the hypothalamus), PVN (paraventricular nucleus of the hypothalamus), LH (lateral hypothalamus), VMN (ventromedial nucleus of the hypothalamus), CcA (central amygdala), and NTS (Nucleus Tractus Solitarius, a longitudinal structure in the medulla). FIG. 2B shows the effect on food intake over a 10 day period for animals administered vehicle ICV, vehicle IP, Aminosterol 1436 at 10 and 40 μg ICV, and Aminosterol 1436 at 5 mg/kg intraperitoneal injection (IP). Finally, FIG. 2C shows the percent change in body weight for the experiment detailed in FIG. 2B, with a decrease in body weight correlating with a decrease in food intake shown in FIG. 2B.

FIG. 3: FIG. 3A shows the plasma concentration (ng/mL) vs time for squalamine lactate after 0.5 mg/kg administered intranasally (IN) in Sprague Dawley® (SD) rats, and FIG. 3C shows the CSF concentration (ng/mL) vs time profile for squalamine lactate following 0.5 mg/kg administered IN to SD rats. Similarly, FIG. 3B shows the plasma concentration (ng/mL) vs time for Aminosterol-1436 (“MSI-1436”) after 0.5 mg/kg administered IN in SD rats, and FIG. 3D shows the CSF concentration (ng/mL) vs time profile for Aminosterol 1436 following 0.5 mg/kg administered IN to SD rats. No squalamine lactate or Aminosterol 1436 was found in CSF following intranasal administration.

FIG. 4: Depicts the hypothalamus in relation to the intercavernous sinus, with the figure clearly showing the intercavernous sinus flowing next to the hypothalamus.

FIG. 5: Depicts the hypothalamus in relation to the cavernous sinus.

FIG. 6: Shows a side-on picture through the nasal cavity showing the turbinates which are highly vascularized.

FIG. 7: Shows the vessels in the nasal cavity, with the cavernous sinus portion of the internal carotid artery (ICA) and the medial basal hypothalamus (MBH), ophthalmic artery (OA), internal carotid artery (ICA), and anterior ethmoidal artery (AEA) identified on the figure.

FIG. 8: Shows the weight change (mean %) following administration to mice of (i) intraperitoneal (IP) administration of 1 mg/kg or 10 mg/kg of Aminosterol 1436, (ii) intranasal (IN) administration of 0.4 mg/kg Aminosterol 1436, or (iii) IN administration of saline control.

FIG. 9: Shows pharmokinetic parameters in a rat of intranasal administration of 0.5 mg/kg as compared to an intravenous bolus of 2/mg/kg (190 μg*hr/ml). Intranasal bioavailability of Aminosterol 1436 (MSI-1436) was found to be about 20%.

FIG. 10: Shows characteristics of autism, including for example, the core autism symptoms of social deficits, language impairment, and repetitive behaviors; associated neurological issues of sleep disorders, mood, anxiety, hyperactivity, seizures, and attention; associated systemic issues of immune dysfunction and GI disorders; and related disorders, such as sleep disorders, mood disorders, anxiety disorders, OCD, and ADHD.

### DETAILED DESCRIPTION OF THE INVENTION

### I. Overview

This invention relates to intranasal compositions comprising a low dose of at least one aminosterol, or a pharmaceutically acceptable salt or derivative thereof. The compositions are formulated for intranasal administration to a subject. The present invention also relates to methods of using of such compositions.

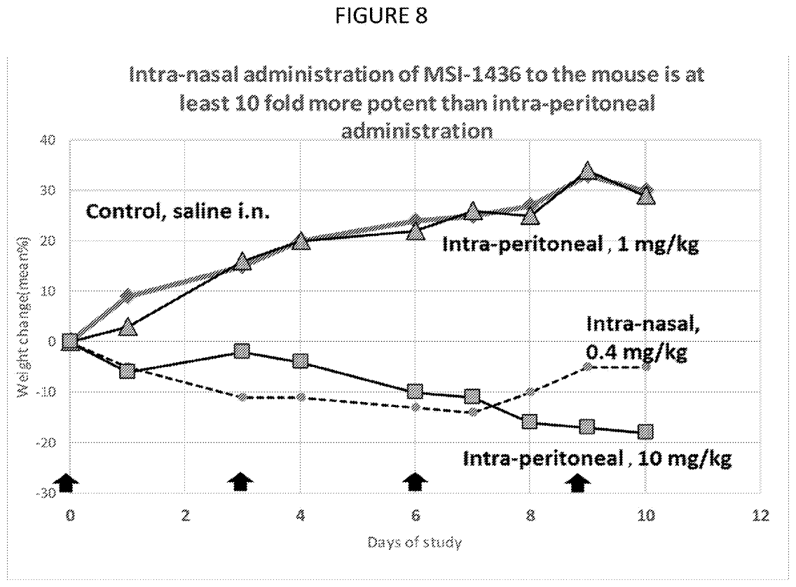

The present invention is based on the discovery of the unexpected and unprecedented activity of an intranasally administered aminosterol in providing a pharmacological effect that is at least 10 fold greater than that observed for non-nasal administration. See e.g., FIG. 8. In particular, FIG. 8 shows that 1 mg/kg Aminosterol 1436 administered IP had no anti-feeding effect on a mouse as the mouse gained weight upon administration, which was the same result seen with saline administration. 10 mg/kg Aminosterol 1436 administered IP showed a marked loss in weight, e.g., a decrease of about 19%, which is consistent with the known pharmacological effect of Aminosterol 1436. Surprisingly, 0.4 mg/kg intranasal (IN) also showed a decrease in weight—of about 5%. Thus, aminosterol dosages which are subtherapeutic when given orally or by injection have pharmacological effect when given IN.

Originally, the inventors theorized that nasal administration of an aminosterol would result in bypassing peripheral blood, as it was thought that following nasal administration, an aminosterol would travel along the olfactory nerve to the spinal fluid, and from there into the brain but not into the peripheral blood. However, experimental results proved this theory incorrect.

Theory: Nasal administration of aminosterol results in drug going from the nose->to the mucosa->to the cerebrospinal fluid->to the brain.

Experimental Proof: Nasal administration of aminosterol results in drug going from the nose->into the capillaries of the nose->drains into the structure behind the nose->then into the hypothalamus.

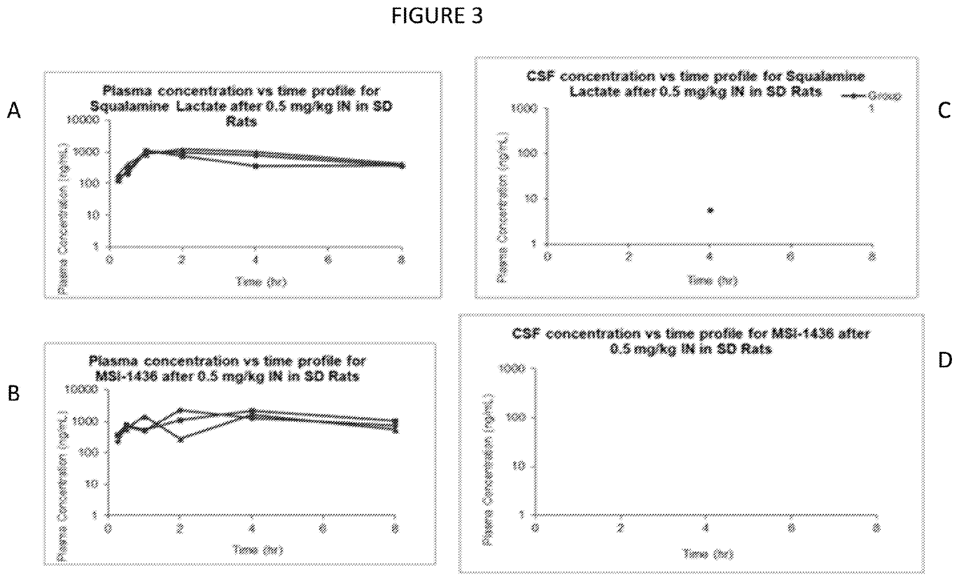

Specifically, as detailed in Example 3, the aminosterols squalamine and Aminosterol 1436 were intranasally administered to rats and spinal fluid levels and blood levels of the aminosterols were measured following administration. See e.g., FIGS. 3A-3D. Surprising, no aminosterol (neither squalamine nor Aminosterol 1436) was detected in the spinal fluid (FIGS. 3C and 3D); but significant levels were detected in the peripheral blood (FIGS. 3A and 3B).

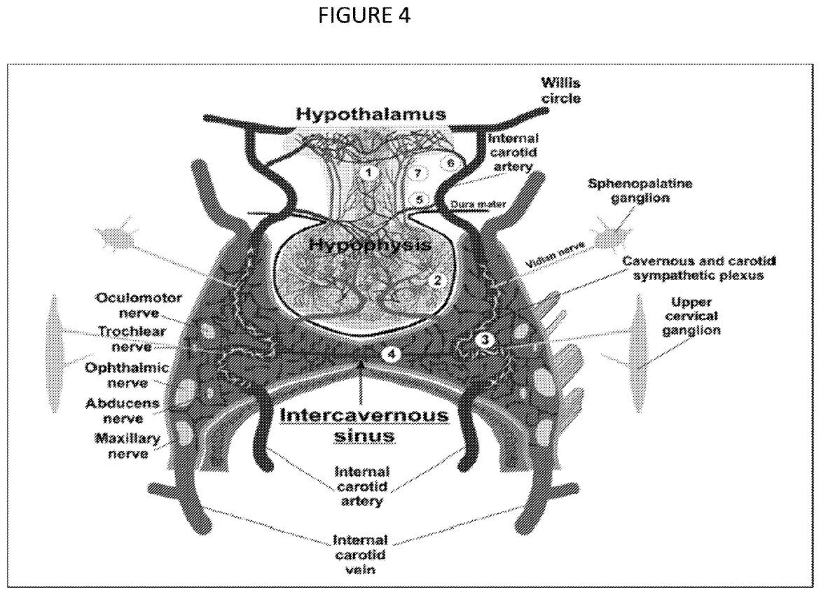

Based on these surprising and unexpected experimental results, it is now theorized that following intranasal administration, aminosterols are not traveling along the sides of the olfactory nerves; rather the drugs are taken up by submucosal blood vessels of the nose (submucosally) and then drained into the cavernous sinus behind the nose. See FIGS. 4, 5, and 6. The arterial and venus blood comprising intranasally administered aminosterol are mixed up in the cavernous sinus, and from there localize to the hypothalamus, which is one of the main sites of neurogenesis.

Importantly, the doses of aminosterol found to have a pharmacological effect following intranasal (IN) administration are significantly lower than those found to be effective following injection, e.g., either intravenous (IV) or intraperitoneal (TP). In particular, the dosage amount that provides a pharmacological effect when administered IN would provide no beneficial pharmacological effect if administered via IV or IP. As an example, and as shown in FIG. 8, a dosage of about 10× the amount used intranasally is required for an aminosterol administered via another route, such as IP or IV, to show pharmacological effect (e.g., an anti-feeding effect). As compared to oral administration, the intranasal aminosterol dosage would be 1/50^{th} the oral dosage to obtain the same pharmacological effect.

Thus, the present invention is an improvement upon earlier disclosures relating to methods of treatment using aminosterols, as the present invention directed to nasal dosage forms of aminosterols surprisingly and unexpectedly enables the use of very low doses of an aminosterol, which previously were not thought to have a pharmacological effect. The intranasal low dose aminosterol dosage forms of the invention can be used in the treatment of any disease amenable to treatment by aminosterols. The intranasal low dose aminosterol dosage forms of the invention provide for the reduction of potential side effects correlated with higher aminosterol dosages as well as allowing for reduced costs. Moreover, intranasal low dose aminosterol compositions allow for targeted drug delivery, as the drug travels directly to the intended site of action, e.g., the hypothalamus of the brain. This delivery method is in contrast to when a drug is administered into the bloodstream, where the drug is then distributed throughout the body, including to portions of the body where drug delivery is undesirable, e.g., the liver. Further, intranasal low dose aminosterol administration allows for greater patience compliance, and in particular for treatment regimens that require repetitive dosing. Further, the methods of the invention do not negatively impact normal health and behavior of the subjects treated.

Prior disclosures relating to methods of treatment using aminosterols reference determining, measuring, and monitoring aminosterol dosage by tracking the amount of the drug appearing in the bloodstream following administration (e.g., by injection). However, the present invention is directed to the discovery that IN-administered aminosterol efficacy is not effected in blood level of drug.

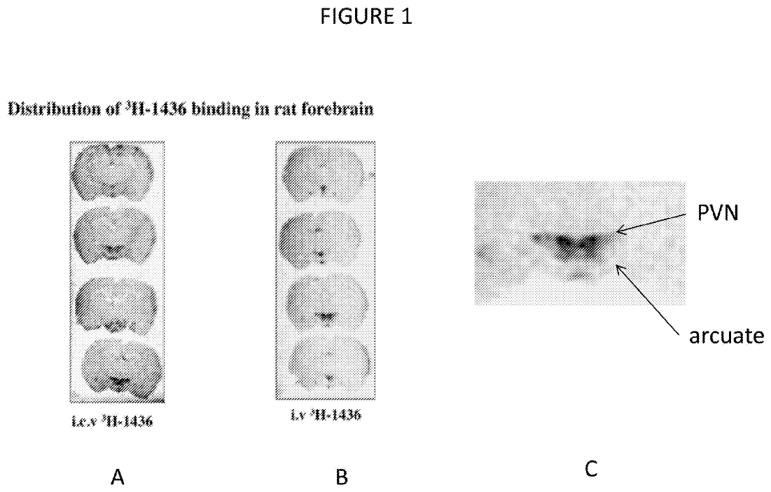

The exact mechanism by which aminosterols, such as Aminosterol 1436 and squalamine, achieve their effect is not known. However, without being bound by theory, it is theorized that the effect of aminosterols such as Aminosterol 1436 and squalamine is likely in part due to the drug's effects on the hypothalamus within the brain of the animal. As seen in FIG. 1, when radioactive Aminosterol 1436 is administered to a rat intravenously via a peripheral vein (IV), or injected directly into the 3^{rd} ventricle of the brain (intracerebroventricular (ICV) administration), the compound accumulates within the centers of the brain that control growth, maturation and senescence.

### Experimental Data

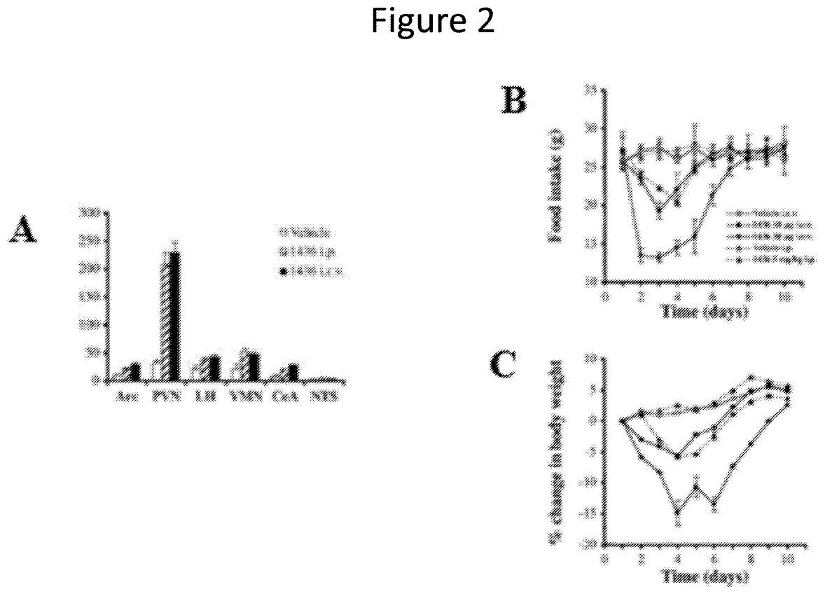

Examples 1 and 2 describe data detailing how regardless of the route of administration, aminosterols such as Aminosterol 1436 localize in the hypothalamus following administration. See FIGS. 1, 2A, and 2B. Based on this result, the impact on a known and readily measured activity of an aminosterol (e.g., food intake for Aminosterol 1436) was determined for different routes of administration, with the results paralleling those seen in Example 1. This data demonstrates that aminosterols such as Aminosterol 1436 act at the level of the hypothalamus following in vivo administration, regardless of the route of administration.

The most dramatic, surprising and unexpected results are detailed in Example 3, where it is shown that IN administered aminosterols, such as Aminosterol 1436 and squalamine, was not absorbed into the CSF, but rather was readily absorbed into the blood. In fact, it was unexpectedly found that IN administration of an aminosterol such as Aminosterol 1436 produced 10 times higher blood levels of Aminosterol 1436 than peripherally injected Aminosterol 1436. See e.g., Example 4 and FIG. 8. In addition, administration of the aminosterol squalamine was also found to result in a similar distribution pattern.

Based on the data detailed in Example 3, it was concluded that administration of aminosterols, such as Aminosterol 1436 and squalamine, results in the drug crossing the nasal epithelium and being absorbed into a very rich submucosal capillary network, from which the drug then drains into the cavernous sinus. Within the cavernous sinus, arterial and venous blood are admixed. Blood from the cavernous sinus is pumped by the internal carotid artery passing through it into the microvasculature of the brain, specifically the microvasculature of the hypothalamus, and more specifically the mesiobasal hypothalamus.

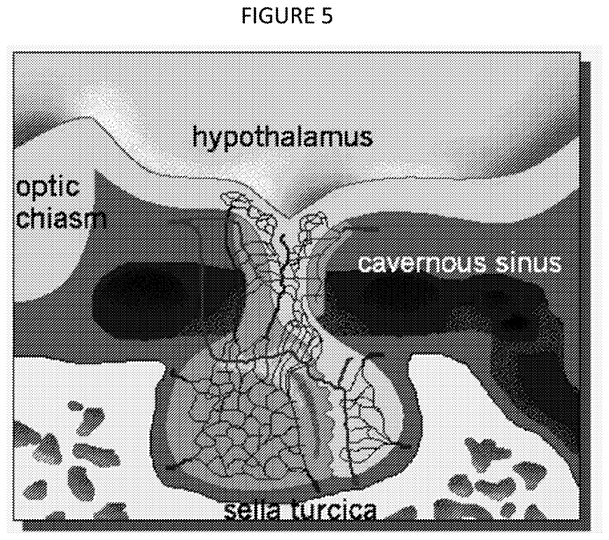

The vascular network in the nasal cavity, the cavernous sinuses right behind the nasal cavity, and the mesiobasal hypothalamus, are all incredibly close to each other (e.g., no more than 1-2 cm apart). See FIG. 4, which clearly shows that the hypothalamus is located very close to the cavernous sinus. A close up of this structure is shown in FIG. 5.

This structure of the brain provides for incredibly rapid transport of an aminosterol directly into the site at which it acts, e.g., the hypothalamus. Thus, minute amounts of an aminosterol compound administered IN are sufficient to produce a pharmacologic effect because they are directly delivered into a tiny compartment very close to the hypothalamus. This was not known prior to the present invention.

Finally, Example 5 describes human clinical data demonstrating that doses of aminosterol which are likely to be efficacious in the human brain were found to be tolerable when administered intranasally.

### II. Intranasal Low Dose Aminosterol Pharmaceutical Compositions

Disclosed herein are intranasal, low dose aminosterol pharmaceutical compositions. The compositions comprise at least one aminosterol, or a pharmaceutically acceptable salt or derivative thereof.

Dosages

The intranasal aminosterol compositions of the invention comprise an aminosterol dosage which would not result in a pharmacological effect if administered via any other route, such as IP, IV, or oral, e.g., the aminosterol dosages are subtherapeutic when given by other routes. For example, Aminosterol 1436 is known to have the pharmacological effects of a reduction in food intake and weight loss (see e.g., FIG. 8). Therefore, in the IN methods of the invention, if the aminosterol is Aminosterol 1436 or a salt or derivative thereof, then if the IN Aminosterol 1436 dosage is administered via another route, such as oral, IP, or IV, then the Aminosterol 1436 dosage will not result in a noticeable reduction in food intake or noticeable weight loss. Similarly, squalamine is known to produce the pharmacological effects of nausea and/or vomiting. Thus, in the IN methods of the invention, if the aminosterol is squalamine or a salt or derivative thereof, then if the IN squalamine dosage is administered via another route, such as oral, IP, or IV, then the squalamine dosage will not result in noticeable nausea and/or vomiting.

The dosage of aminosterol administered can range from about 0.001 to about 6 mg, or any amount in-between these two values. For example, the low dosage of an aminosterol or a salt or derivative thereof can be about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.25, about 1.3, about 1.4, about 1.5, about 1.6, about 1.7, about 1.75, about 1.8, about 1.9, about 2, about 2.1, about 2.2, about 2.25, about 2.3, about 2.4, about 2.5, about 2.6, about 2.7, about 2.75, about 2.8, about 2.9, about 3, about 3.1, about 3.2, about 3.25, about 3.3, about 3.4, about 3.5, about 3.6, about 3.7, about 3.75, about 3.8, about 3.9, about 4, about 4.1, about 4.2, about 4.25, about 4.3, about 4.4, about 4.5, about 4.6, about 4.7, about 4.75, about 4.8, about 4.9, about 5 mg, about 5.1, about 5.2, about 5.25, about 5.3, about 5.4, about 5.5, about 5.6, about 5.7, about 5.75, about 5.8, about 5.9, or about 6 mg.

The delivered volume per spray for a liquid nasal spray can be, for example, about 0.2 mls. A dose can be delivered in multiple nasal sprays (e.g., 2, 3, or 4). In other embodiments, the delivered volume per spray for a nasal spray can be about 0.025, about 0.03, about 0.04, about 0.05, about 0.06, about 0.07, about 0.08, about 0.09, about 0.1, about 0.15, about 0.16, about 0.17, about 0.18, about 0.19, about 0.2, about 0.21, about 0.22, about 0.23, about 0.24, about 0.25, about 0.26, about 0.27, about 0.28, about 0.29, or about 0.3 mls. Typical metered dose nasal sprays deliver about 0.025 to about 0.2 mls per spray. The total volume of the nasal cavity ranges from 13 mL to 20 mL.

In addition, examples of such aminosterol low dosages include, but are not limited to, about 0.001 to 4 mg/kg, or any amount in-between these two values. For example, the low dosage of an aminosterol can be about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.25, about 1.3, about 1.4, about 1.5, about 1.6, about 1.7, about 1.75, about 1.8, about 1.9, about 2, about 2.1, about 2.2, about 2.25, about 2.3, about 2.4, about 2.5, about 2.6, about 2.7, about 2.75, about 2.8, about 2.9, about 3, about 3.1, about 3.2, about 3.25, about 3.3, about 3.4, about 3.5, about 3.6, about 3.7, about 3.75, about 3.8, about 3.9, about 4 mg/kg.

Aminosterols

U.S. Pat. No. 6,962,909, entitled “Treatment of neovascularization disorders with squalamine,” discloses various aminosterols, and this disclosure is specifically incorporated by reference with respect to its teaching of aminosterol compounds. Any aminosterol known in the art, including those described in U.S. Pat. No. 6,962,909, can be used in the disclosed compositions. In some embodiments, the aminosterol present in the compositions of the invention is Aminosterol 1436, squalamine, or a combination thereof.

For instance, useful aminosterol compounds comprise a bile acid nucleus and a polyamine, attached at any position on the bile acid, such that the molecule exhibits a net positive charge contributed by the polyamine.

Thus, in some embodiments, the disclosed methods comprise intranasally administering a therapeutically effective amount of one or more aminosterols having the chemical structure of Formula I:

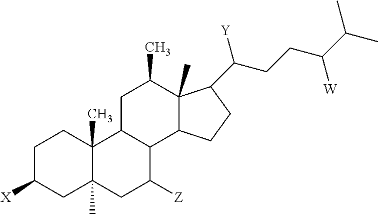

wherein,

W is 24S —OSO₃ or 24R—OSO₃;

X is 3β-H₂N—(CH₂)₄—NH—(CH₂)₃—NH— or 3α-H₂N—(CH₂)₄—NH—(CH₂)₃—NH—;

Y is 20R—CH₃; and

Z is 7a or 7β —OH.

In another embodiment of the invention, the aminosterol is one of the naturally occurring aminosterols (1-8) isolated from Squalus acanthias:

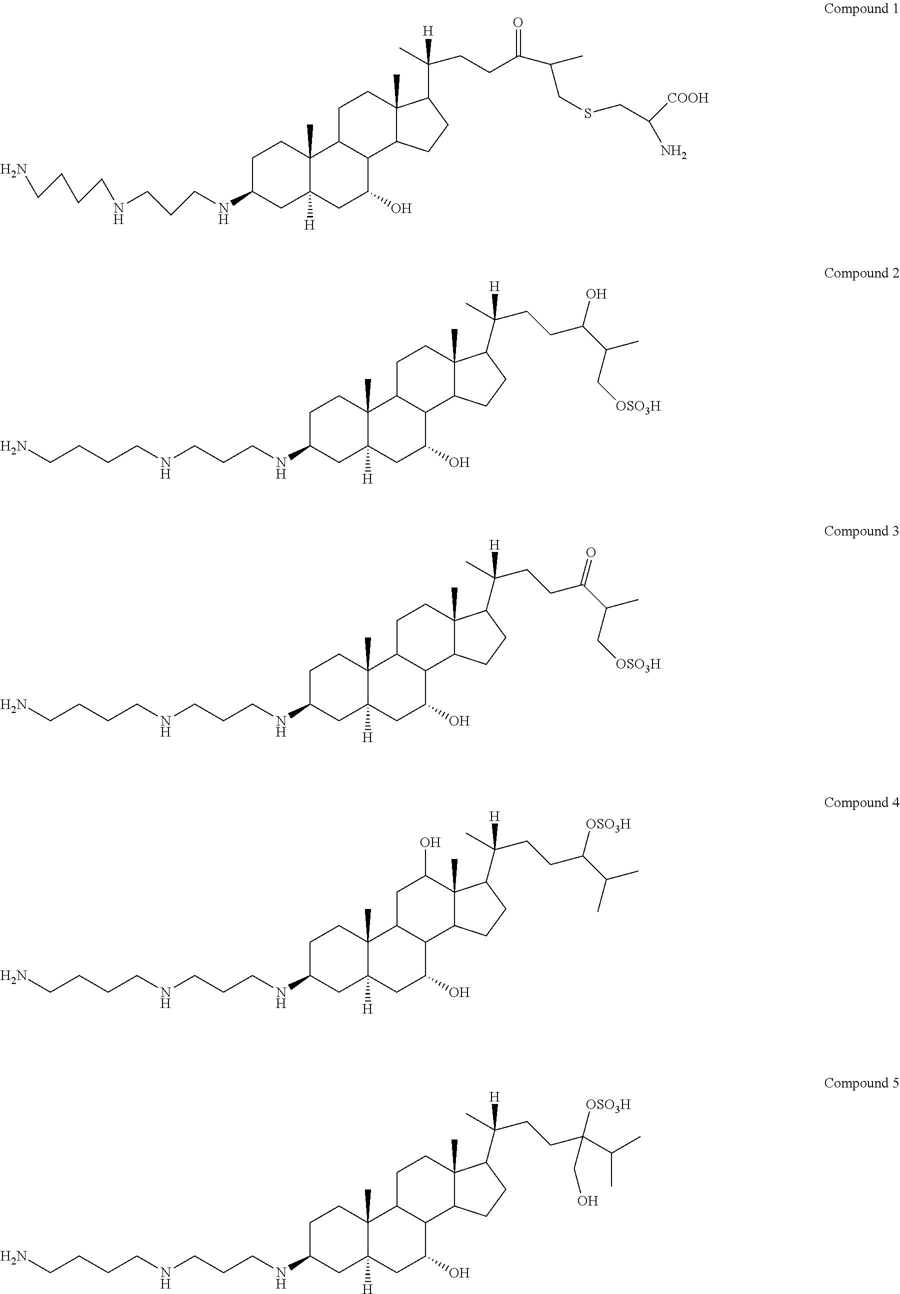

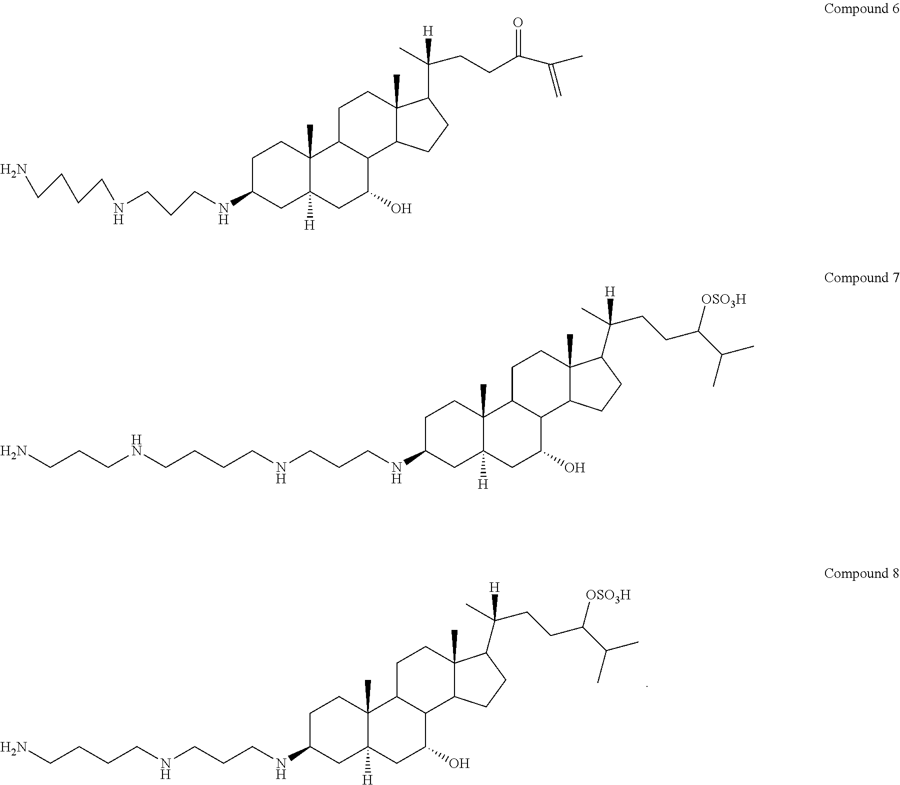

In one aspect of the invention, the aminosterol is Aminosterol 1436 or a salt or derivative thereof. In another embodiment the aminosterol is squalamine or a salt or derivative thereof.

Variants or derivatives of known aminosterols, such as squalamine, Aminosterol 1436, or an aminosterol isolated from Squalus acanthias, may be used in the disclosed compositions and methods.

In one embodiment, the aminosterol is Aminosterol 1436 or a squalamine isomer. In yet another embodiment of the invention, the aminosterol is a derivative of squalamine or another naturally occurring aminosterol modified through medical chemistry to improve biodistribution, ease of administration, metabolic stability, or any combination thereof. In another embodiment, the squalamine or aminosterol is modified to include one or more of the following: (1) substitutions of the sulfate by a sulfonate, phosphate, carboxylate, or other anionic moiety chosen to circumvent metabolic removal of the sulfate moiety and oxidation of the cholesterol side chain; (2) replacement of a hydroxyl group by a non-metabolizable polar substituent, such as a fluorine atom, to prevent its metabolic oxidation or conjugation; and (3) substitution of various ring hydrogen atoms to prevent oxidative or reductive metabolism of the steroid ring system.

In yet another embodiment, the aminosterol comprises a sterol nucleus and a polyamine, attached at any position on the sterol, such that the molecule exhibits a net charge of at least +1, the charge being contributed by the polyamine.

In yet another embodiment, the aminosterol comprises a bile acid nucleus and a polyamine, attached at any position on the bile acid, such that the molecule exhibits a net positive charge being contributed by the polyamine.

In some embodiments, the compositions used in the methods of the invention comprise: (a) at least one pharmaceutical grade aminosterol; and optionally (b) at least one phosphate selected from the group consisting of an inorganic phosphate, an inorganic pyrophosphate, and an organic phosphate. In some embodiments, the aminosterol is formulated as a weakly water soluble salt of the phosphate. In some embodiments, the phosphate is an inorganic polyphosphate, and the number of phosphates can range from about 3 (tripolyphosphate) to about 400, or any number in-between these two values. In other embodiments, the phosphate is an organic phosphate which comprises glycerol 2 phosphates.

In some embodiments, the aminosterol is selected from the group consisting of: (a) squalamine or a pharmaceutically acceptable salt or derivative thereof, (b) a squalamine isomer; (c) a phosphate salt of squalamine; (d) Aminosterol 1436; (e) an isomer of Aminosterol 1436; (f) a phosphate salt of Aminosterol 1436; (g) a synthetic aminosteorl; (h) an aminosterol comprising a sterol or bile acid nucleus and a polyamine, attached at any position on the sterol or bile acid, such that the molecule exhibits a net charge of at least +1, the charge being contributed by the polyamine; (i) an aminosterol which is a derivative of squalamine modified through medical chemistry to improve biodistribution, ease of administration, metabolic stability, or any combination thereof, (j) an aminosterol modified to include one or more of the following: (i) substitutions of the sulfate by a sulfonate, phosphate, carboxylate, or other anionic moiety chosen to circumvent metabolic removal of the sulfate moiety and oxidation of the cholesterol side chain; (ii) replacement of a hydroxyl group by a non-metabolizable polar substituent, such as a fluorine atom, to prevent its metabolic oxidation or conjugation; and (iii) substitution of various ring hydrogen atoms to prevent oxidative or reductive metabolism of the steroid ring system; (k) an aminosterol that can inhibit the formation of actin stress fibers in endothelial cells stimulated by a ligand known to induce stress fiber formation, having the chemical structure of Formula I (above); or (l) any combination thereof.

In some embodiments, the methods of the invention can employ a formulation of Aminosterol 1436 (Zasloff, Williams et al. 2001) as an insoluble salt of phosphate, polyphosphate, or an organic phosphate ester.

Any pharmaceutically acceptable salt of an aminosterol can be used in the compositions and methods of the invention. For example, a phosphate salt or buffer, free base, succinate, phosphate, mesylate or other salt form associated with low mucosal irritation can be utilized in the methods and compositions of the invention.

Composition Components

In some embodiments, a pharmaceutical composition disclosed herein comprises an aqueous carrier.

In some embodiments, a pharmaceutical composition disclosed herein further comprises a buffer.

In some embodiments, a pharmaceutical composition disclosed herein further comprises a diluent, such as lactose.

In some embodiments, a pharmaceutical composition disclosed herein further comprises a simple polyol compound, such as glycerine. Other examples of polyol compounds include sugar alcohols. In some embodiments, a pharmaceutical composition disclosed herein comprises an aqueous carrier and glycerine at about a 2:1 ratio.

The formulations may conveniently be presented in unit dosage form and may be prepared by any of the methods well known in the art of pharmacy in regards to intranasal administration. An exemplary dosage form is a nasal spray. A nasal spray is designed to deliver drug to the upper nasal cavity, and can be a liquid or powder formulation, and in a dosage form such as an aerosol, liquid spray, or powder.

In some embodiments, a pharmaceutical composition disclosed herein is a nasal spray, comprising a dry powder, liquid suspension, liquid emulsion, or other suitable nasal dosage form.

The pharmaceutical compositions discloses herein may comprise or be housed in any nasal spray device known in the art, such as a needle-free device or a “ready-to-use” device, wherein minimal or no manipulations are required to use the device and administer the composition into a nostril. The nasal spray device, in some aspects, may be a disposable device suitable for placement in household trash and not requiring formal hazardous waste disposal as is true for needle-based delivery. In one aspect, the device used allows for administration of a volume of from about 50 μL to about 250 μL or from about 75 μL to about 200 μL or from about 80 μL to about 120 μL or from about 90 μL to about 110 μL or from about 100 μL to about 150 μL or about 100 μL or about 180 μL to about 220 μL or about 200 μL.

The disclosed nasal spray device, as set forth above, is intended for use by both medical and non-medical personnel. In particular, the device may have one or more features selected from being single-use, needle-free, ready-to-use, disposable, and combinations thereof. The device may be configured to administer the disclosed compositions as a single spray per naris. The device may comprise one or more unit dose containers, each container delivering about one 100 μL spray with an appropriate amount of an aminosterol. In other aspects, the devices may be modified to deliver amounts of between about 50 μL to about 200 L spray, and may utilize solutions of varying concentration

Intranasal delivery-enhancing agents may be employed which enhance delivery of an aminosterol into or across a nasal mucosal surface. For passively absorbed drugs, the relative contribution of paracellular and transcellular pathways to drug transport depends upon the pKa, partition coefficient, molecular radius and charge of the drug, the pH of the luminal environment in which the drug is delivered, and the area of the absorbing surface. The intranasal delivery-enhancing agent of the present invention may be a pH control agent.

Within the mucosal delivery formulations and methods of the invention, the aminosterol may be combined or coordinately administered with a suitable carrier or vehicle for mucosal delivery. As used herein, the term “carrier” means pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories can be found in the U.S. Pharmacopeia National Formulary, 1857-1859, and (1990). Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other non toxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art. Examples of filling agents include lactose monohydrate, lactose anhydrous, and various starches; examples of binding agents include various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil® 200, talc, stearic acid, magnesium stearate, calcium stearate, and silica gel. Examples of sweeteners may include any natural or artificial sweetener, such as sucrose, xylitol, sodium saccharin, cyclamate, aspartame, and acesulfame. Examples of flavoring agents are Magnasweet® (trademark of MAFCO), bubble gum flavor, and fruit flavors, and the like. Examples of preservatives include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

In some embodiments, the administration of a pharmaceutical composition disclosed herein results in a Tₘₐₓ in a subject from about 0.1 hours to about 0.5 hours, or from about 0.3 hours to about 0.5 hours, or about 0.2 hours, or about 0.3 hours, or about 0.4 hours, about 0.5 hours, about 1 hour, about 1.5 hours, about 2 hours, about 3 hours, about 4 hours, about 5 hours, about 6 hours, about 7 hours, or about 8 hours after intranasal administration.

The pharmaceutical composition comprising an aminosterol derivatives or salts thereof will be formulated and dosed in a fashion consistent with good medical practice, taking into account the clinical condition of the individual patient, the method of administration, the scheduling of administration, and other factors known to practitioners. The “effective amount” for purposes herein is thus determined by such considerations.

Dosing Period

The pharmaceutical composition comprising an aminosterol or a derivative or salt thereof can be intranasally administered for any suitable period of time, including as a maintenance dose for a prolonged period of time. Dosing can be done on an as needed basis using any pharmaceutically acceptable dosing regimen. For example, dosing can be once or twice daily, once every other day, once every three days, once every four days, once every five days, once every six days, once a week, or divided over multiple time periods during a given day (e.g., twice daily). The dosing schedule may include administration during the morning, midday, or during the evening, or a combination thereof.

In other embodiments, the composition can be administered: (1) as a single dose, or as multiple doses over a period of time; (2) at a maintenance dose for an indefinite period of time; (3) once, twice or multiple times; (4) daily, every other day, every 3 days, weekly, or monthly; (5) for a period of time such as about 1, about 2, about 3, or about 4 weeks, about 1, about 2, about 3, about 4, about 5, about 6, about 7, about 8, about 9, about 10, about 11, or about 12 months, about 1 year, about 1.5 years, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, or about 25 years, or (6) any combination of these parameters, such as daily administration for 6 months, weekly administration for 1 or more years, etc.

Exemplary dosing regimens include, but are not limited to: Initiating with a “low” initial daily dose, and gradually increasing the daily dose until a dose is reached that elicits evidence of a measurable impact, or other indicia of desirable effects. Another exemplary dosing regimen includes: Initiating with a “high” initial dose, and reducing the subsequent daily dosing to that required to elicit a desirable response.

Yet another exemplary dosing regimen includes periodic dosing, where an effective dose can be delivered once every about 1, about 2, about 3, about 4, about 5, about 6 days, or once weekly.

Any pharmaceutical used for therapeutic administration can be sterile. Sterility is readily accomplished by for example filtration through sterile filtration membranes (e.g., 0.2 micron membranes). Any pharmaceutically acceptable sterility method can be used in the compositions of the invention.

Kits

Formulations or compositions of the invention may be packaged together with, or included in a kit along with instructions or a package insert. Such instructions or package inserts may address recommended storage conditions, such as time, temperature and light, taking into account the shelf-life of the aminosterol or derivatives or salts thereof. Such instructions or package inserts may also address the particular advantages of the aminosterol or derivatives or salts thereof, such as the ease of storage for formulations that may require use in the field, outside of controlled hospital, clinic or office conditions.

The invention also provides a pharmaceutical pack or kit comprising one or more containers filled with one or more pharmaceutical compositions disclosed herein. The kits may include, for instance, containers filled with an appropriate amount of a pharmaceutical composition, either as a powder, to be dissolved, or as a sterile solution, in addition to the aminosterol or a derivative or salt thereof. Associated with such container(s) can be a notice in the form prescribed by a governmental agency regulating the manufacture, use or sale of pharmaceuticals or biological products, which notice reflects approval by the agency of manufacture, use or sale for human administration. In addition, the aminosterol or a derivative or salt thereof may be employed in conjunction with other therapeutic compounds.

In other aspects, a kit comprising a nasal spray device as described herein is disclosed. In one aspect, the kit may comprise one or more devices as disclosed herein, comprising a disclosed low dose aminosterol composition, wherein the device is sealed within a container sufficient to protect the device from atmospheric influences. The container may be, for example, a foil, or plastic pouch, particularly a foil pouch, or heat sealed foil pouch. Suitable containers sufficient to adequately protect the device will be readily appreciated by one of skill in the art.

In one aspect, the kit may comprise one or more devices as disclosed herein, wherein the device may be sealed within a first protective packaging, or a second protective packaging, or a third protective packaging, that protects the physical integrity of the nasal spray product. One or more of the first, second, or third protective packaging may comprise a foil pouch. The kit may further comprise instructions for use of the device. In one aspect, the kit contains two or more nasal spray devices.

In one aspect, the kit may comprise a device as disclosed herein, and may further comprise instructions for use. In one aspect, the instructions may comprise visual aid/pictorial and/or written directions to an administrator of the device. The directions may include the steps of (a) placing the individual on their back; (b) inserting a first sprayer into the individual's nostril; (c) aiming the nozzle towards the side of the individual's nose and away from the center of the nose; (d) pressing a plunger of the device firmly with the thumb of the administrator; and (e) repeating steps b through d with a second sprayer in the second nostril of the individual's nose.

The kit may comprise one, two, three or more nasal spray devices and instructions for use; wherein each nasal spray device comprises from about 80 μL to about 120 μL of a disclosed nasal spray, or from about 90 μL to about 110 μL of a disclosed nasal spray.

### III. Methods of Use of Intranasal Pharmaceutical Compositions

The disclosed intranasal compositions can be used to treat a range of subjects, including human and non-human animals, including mammals, as well as immature and mature animals, including human children and adults. The human subject to be treated can be an infant, toddler, school-aged child, teenager, young adult, adult, or elderly patient.

The conditions, diseases, or indications that can be treated using the disclosed intranasal compositions include any condition, disease, or indication amenable to treatment with an aminosterol. Examples of such conditions, diseases, or indications are described below, but the compositions of the invention are not limited to treatment of the exemplified conditions. Conditions, diseases, or indications amenable to treatment with the compositions of the invention are also described in U.S. Pat. Nos. 9,867,835 and 8,729,058, both for “Methods and compositions for treating and preventing viral infections;” WO 2015/00195 and US 2015/0368290, both for “Methods and compositions for stimulation of the intestinal enteroendocrine system for treating diseases or conditions related to the same;” and U.S. Pat. Nos. 8,716,270 and 7,981,876, both for “Polymorphic and Amorphous Salt Forms of Squalamine Dilactate,” the disclosures of which are specifically incorporated by reference.

A. Combination Aminosterol Treatment

The invention encompasses methods of treatment using an intranasal low dose aminosterol composition according to the invention, combined with administration of the same or a different aminosterol via a different administration route, such as oral or injection (e.g., IP, IV, IM).

For combination methods of treatment, the dosage of aminosterol present in the oral or injectable composition is higher than that utilized for the IN low dose aminosterol dosage form. For example, the second non-IN aminosterol dosage form can comprise an aminosterol dosage ranging from, for oral administration, about 10 mg to about 1000 mg, or any amount in-between these two values. For example, the oral dosage form can comprise the following amount of an aminosterol: about 15 mg, about 20, about 25, about 30, about 35, about 40, about 45, about 50, about 55, about 60, about 65, about 70, about 75, about 80, about 85, about 90, about 95, about 100, about 105, about 110, about 150, about 120, about 125, about 130, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 275, about 300, about 325, about 350, about 375, about 400, about 425, about 450, about 475, about 500 mg, about 525, about 550, about 575, about 600, about 625, about 650, about 675, about 700, about 725, about 750, about 775, about 800, about 825, about 850, about 875, about 900, about 925, about 950, about 975, or about 1000 mg.

For injectable dosage forms, the non-IN dosage form can comprise an aminosterol at a dosage of, for example, about 0.1 to about 20 mg/kg body weight. In other embodiments, the effective daily dosing amount is about 0.1, about 0.5, about 1, about 2, about 3, about 4, about 5, about 6, about 7, about 8, about 9, about 10, about 11, about 12, about 13, about 14, about 15, about 16, about 17, about 18, about 19, or about 20 mg/kg body weight.

In one embodiment of a combination method, the aminosterol administered intranasally is aminosterol 1436 or a salt or derivative thereof, and the aminosterol administered orally is squalamine or a salt or derivative thereof.

B. Treatment and/or Prevention of Neurodegeneration

The intranasal low dose aminosterol compositions of the invention can be used to treat and/or prevent neurodegeneration in a subject in need. For example, the intranasal low dose aminosterol compositions can be used to treat and/or prevent conditions including but not limited to (i) age-related neurodegeneration, (ii) age-related neurodegeneration correlated with age-related dementia, or (iii) neurodegeneration correlated with a neurodisease, such as Parkinson's disease (PD), supranuclear palsy, multi-system atrophy, Parkinsonism, Alzheimer's disease, frontotemporal dementia, amyotrophic lateral sclerosis (ALS), Huntington's Disease, schizophrenia, Friedreich's ataxia, Multiple sclerosis (MS), Lewy Body dementia or disease, spinal muscular atrophy, frontotemporal dementia, progressive nuclear palsy, Guadeloupian Parkinsonism, spinocerebellar ataxia, or vascular dementia.

The method comprises comprising administering to a subject in need an intranasal low dose aminosterol pharmaceutical composition according to the invention.

As detailed above and in the examples, administration of the intranasal low dose aminosterol compositions of the invention triggers neurogenesis. Neurogenesis is the process by which nervous system cells, known as neurons, are produced by neural stem cells (NSC)s. Typically, neurogenesis is most active during embryonic development, and is responsible for producing all the various types of neurons of the organism, but continues throughout adult life in a variety of organisms.

By stimulating neurogenesis, the intranasal low dose aminosterol compositions of the invention trigger the differentiation of nerve cells into neurons. This promotes migration of the neurons along established neural pathways in the brain; for example, the anterior and posterior pathways. A neural pathway is the connection formed by axons that project from neurons to make synapses onto neurons in another location, to enable a signal to be sent from one region of the nervous system to another. Neurons are connected by a single axon, or by a bundle of axons known as a nerve tract, or fasciculus. Shorter neural pathways are found within grey matter in the brain, whereas longer projections, made up of myelinated axons, constitute white matter.

Every 2-3 weeks the neurons of the olfactory bulb are completely repopulated, and this repopulation ensures that humans have a good sense of smell throughout life. Thus, in one embodiment of the invention, the intranasal low dose aminosterol compositions, which trigger neurogenesis, can be used to treat neurodegeneration in a subject that has resulted in a loss of smell (anosmia (complete loss of smell) or hyposmia (partial loss of smell)), with a result of either complete or partial restoration of the sense of smell. Examples of patient populations that may suffer from anosmia or hyposmia include, but are not limited to, subjects with head trauma and subjects that may later develop Parkinson's disease or other neurodiseases.

Thus, in one embodiment of the invention, encompassed are methods of treating a subject suffering from anosmia or hyposmia, comprising administering an intranasal low dose aminosterol composition according to the invention. The method can result in improving the subject's sense of smell. For example, the improvement can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

The intranasal low dose aminosterol compositions of the invention will stimulate neurogeneration where neurogeneration already exists, which means that the compositions of the invention will result in increased neurogeneration. Thus, if a subject has inflammation in their brain as a result of a neurodisease, then administration of the intranasal low dose aminosterol compositions of the invention can reverse the inflammation—as well as disease symptoms—by increasing the number of new cells being brought into tissues; this is because neurodegeneration is stopped by adding more cells than are dying.

Age-related neurodegeneration is a significant unsolved problem and challenge. The number of people over 60 years is expected to rise from 841 million in 2013 to more than 2 billion in 2050. As populations get older, age-related neurodegenerative diseases such as Alzheimer's Disease (AD) and Parkinson's Disease (PD) have become more common.

Research to date regarding neurodegenerative diseases has resulted in only modest success. For AD, PD, and ALS, researchers have looked at everything from mis-folded proteins to infectious agents. However, none of the current treatments alters the course of these age-related diseases. They remain incurable.

The World Health Organisation looked at 23 low-to middle-income nations and estimated that their combined loss in economic output between 2006 and 2015 due to age-related diseases was USD84 billion, and the global cost of AD alone in 2010 was estimated at USD604 billion. Wimo et al., “The worldwide economic impact of dementia 2010,” Alzheimers Dement., 9: 1-11 (2013).

Alzheimer's disease (AD) is a chronic neurodegenerative disease that usually starts slowly and worsens over time. It is the cause of 60-70% of cases of dementia. As the disease advances, symptoms can include problems with language, disorientation, mood swings, loss of motivation, not managing self care, and behavioral issues. As a person's condition declines, they often withdraw from family and society. Gradually, bodily functions are lost, ultimately leading to death. Although the speed of progression can vary, the typical life expectancy following diagnosis is 3 to 9 years. In 2015, there were approximately 29.8 million people worldwide with AD. It most often begins in people over 65 years of age, although 4% to 5% of cases are early-onset Alzheimer's. It affects about 6% of people 65 years and older. In 2015, dementia resulted in about 1.9 million deaths.

Parkinson's Disease (PD) is the second most common age-related neurodegenerative disease after AD. Reeve et al. (2014). PD affects over 1% of the population over the age of 60, which in the US equates to over 500,000 individuals, while in individuals over the age of 85 this prevalence reaches 5%, highlighting the impact that advancing age has on the risk of developing this condition.

Lewy body dementia (LBD) is a disease associated with abnormal deposits of a protein called alpha-synuclein in the brain. These deposits, called Lewy bodies, affect chemicals in the brain whose changes, in turn, can lead to problems with thinking, movement, behavior, and mood. frontotemporal dementia (FTD) is a group of related conditions resulting from the progressive degeneration of the temporal and frontal lobes of the brain. These areas of the brain play a significant role in decision-making, behavioral control, emotion and language. Finally, vascular dementia is a decline in thinking skills caused by conditions that block or reduce blood flow to the brain, depriving brain cells of vital oxygen and nutrients.

Huntington's disease (HD) is a fatal genetic disorder that causes the progressive breakdown of nerve cells in the brain. It deteriorates a person's physical and mental abilities during their prime working years and has no cure. Full-time care is required in the later stages of the disease. Symptoms of Huntington's disease most commonly become noticeable between the ages of 35 and 44 years, but they can begin at any age from infancy to old age. The most characteristic initial physical symptoms are jerky, random, and uncontrollable movements called chorea. Suicide is the cause of death in about 9% of cases. Death typically occurs 15 to 20 years from when the disease was first detected.

Progressive supranuclear palsy, also called Steele-Richardson-Olszewski syndrome, is an brain disorder that causes serious problems with walking, balance and eye movements. The disorder results from deterioration of cells in areas of the brain that control body movement and thinking. There is no known cure for PSP and management is primarily supportive.

Multiple system atrophy (MSA) is a progressive neurodegenerative disorder characterized by a combination of symptoms that affect both the autonomic nervous system (the part of the nervous system that controls involuntary action such as blood pressure or digestion) and movement. MSA, also known as Shy-Drager syndrome, is a neurodegenerative disorder characterized by tremors, slow movement, muscle rigidity, and postural instability (collectively known as parkinsonism) due to dysfunction of the autonomic nervous system, and ataxia. This is caused by progressive degeneration of neurons in several parts of the brain including the substantia nigra, striatum, inferior olivary nucleus, and cerebellum. There is no known cure for MSA and management is primarily supportive.

Frontotemporal dementia (FTD) is a group of related conditions resulting from the progressive degeneration of the temporal and frontal lobes of the brain. These areas of the brain play a significant role in decision-making, behavioral control, emotion and language. The frontotemporal dementias (FTD) encompass six types of dementia involving the frontal or temporal lobes. They are: behavioral variant of FTD, semantic variant primary progressive aphasia, nonfluent agrammatic variant primary progressive aphasia, corticobasal syndrome, progressive supranuclear palsy, and FTD associated with motor neuron disease. Currently, there is no cure for FTD.

Vascular dementia, also known as multi-infarct dementia (MID) and vascular cognitive impairment (VCI), is dementia caused by problems in the supply of blood to the brain, typically a series of minor strokes, leading to worsening cognitive decline that occurs step by step. Risk factors for vascular dementia include age, hypertension, smoking, hypercholesterolemia, diabetes mellitus, cardiovascular disease, and cerebrovascular disease. Other risk factors include geographic origin, genetic predisposition, and prior strokes.

Amyotrophic lateral sclerosis (ALS), also known as motor neuron disease (MND), or Lou Gehrig's disease, is a specific disease which causes the death of neurons controlling voluntary muscles. ALS is characterized by stiff muscles, muscle twitching, and gradually worsening weakness due to muscles decreasing in size. This results in difficulty speaking, swallowing, and eventually breathing. The cause is not known in 90% to 95% of cases. The remaining 5-10% of cases are genetic. The underlying mechanism involves damage to both upper and lower motor neurons. No cure for ALS is known. The disease can affect people of any age, but usually starts around the age of 60 and in inherited cases around the age of 50. The average survival from onset to death is 2 to 4 years, although about 10% survive longer than 10 years.

Multiple sclerosis (MS) is a demyelinating disease in which the insulating covers of nerve cells in the brain and spinal cord are damaged. This damage disrupts the ability of parts of the nervous system to communicate, resulting in a range of signs and symptoms, including physical, mental, and sometimes psychiatric problems. Specific symptoms can include double vision, blindness in one eye, muscle weakness, trouble with sensation, or trouble with coordination. MS takes several forms, with new symptoms either occurring in isolated attacks (relapsing forms) or building up over time (progressive forms). Between attacks, symptoms may disappear completely; however, permanent neurological problems often remain, especially as the disease advances. While the cause is not clear, the underlying mechanism is thought to be either destruction by the immune system or failure of the myelin-producing cells. Proposed causes for this include genetics and environmental factors such as being triggered by a viral infection. There is no known cure for MS. Life expectancy is on average 5 to 10 years lower than that of an unaffected population. MS is the most common immune-mediated disorder affecting the central nervous system. In 2015, about 2.3 million people were affected globally, and in 2015 about 18,900 people died from MS, up from 12,000 in 1990.

Spinal muscular atrophy (SMA) is an inherited neuromuscular disorder characterized by loss of motor neurons and progressive muscle wasting, often leading to early death. The disorder is caused by a genetic defect in the SMN1 gene, which encodes SMN, a protein necessary for survival of motor neurons. Lower levels of the protein results in loss of function of neuronal cells in the anterior horn of the spinal cord and subsequent system-wide atrophy of skeletal muscles. SMA is the most common genetic cause of infant death. In December 2016, nusinersen became the first approved drug to treat SMA while several other compounds remain in clinical trials.

Friedreich's ataxia is an autosomal recessive inherited disease that causes progressive damage to the nervous system. It manifests in initial symptoms of poor coordination such as gait disturbance; it can also lead to scoliosis, heart disease and diabetes, but does not affect cognitive function. The ataxia of Friedreich's ataxia results from the degeneration of nervous tissue in the spinal cord, in particular sensory neurons essential (through connections with the cerebellum) for directing muscle movement of the arms and legs. The spinal cord becomes thinner and nerve cells lose some of their myelin sheath (the insulating covering on some nerve cells that helps conduct nerve impulses).

Progression of neurodegeneration can be measured using well known techniques. For example, an electroencephalogram (EEG) can be used as a biomarker for the presence and progression of a neurodegenerative disease. S. Morairty, “Detecting Neurodegenerative Diseases Before Damage Is Done,” SRI International (Jul. 26, 2013) (https://www.sri.com/blog/detecting-neurodegenerative-diseases). Another exemplary technique that can be used to measure progression of neurodegeneration of MRI. Rocca et al., “The Role of T1-Weighted Derived Measures of Neurodegeneration for Assessing Disability Progression in Multiple Sclerosis,” Front Neurol., 8:433 (Sep. 4, 2017).

A variety of neuroimaging techniques may be useful for the early diagnosis and/or measurement of progression of neurodegenerative disorders. Examples of such techniques include but are not limited to neuroimaging, functional MRI, structural MRI, diffusion tensor imaging (DTI) (including for example diffusion tensor measures of anatomical connectivity), [18F]fluorodeoxyglucose (FDG) PET, agents that label amyloid, [18F]F-dopa PET, radiotracer imaging, volumetric analysis of regional tissue loss, specific imaging markers of abnormal protein deposition (e.g., for AD progression), multimodal imaging, and biomarker analysis. Jon Stoessl, “Neuroimaging in the early diagnosis of neurodegenerative disease,” Transl. Neurodegener., 1: 5 (2012). Combinations of these techniques can also be used to measure disease progression.

For example, structural MRI can be used to measure atrophy of the hippocampus and entorhinal cortex in AD, as well as involvement of the lateral parietal, posterior superior temporal and medial posterior cingulate cortices. In frontotemporal dementias (FTD), structural MRI can show atrophy in frontal or temporal poles. DTI can be used to show abnormal white matter in the parietal lobes of patients with dementia with Lewy bodies (DLB) as compared to AD. Functional MRI may reveal reduced frontal but increased cerebellar activation during performance of a working memory task in FTD compared to AD. In another example, [18F]fluorodeoxyglucose (FDG) PET can show reduced glucose metabolism in parietotemporal cortex in AD. Id.

In one embodiment of the invention, the progression or onset of a neurodegenerative disorder is slowed or prevented over a defined time period, following administration of an intranasal low dose aminosterol composition of the invention to a subject in need, as measured by a medically-recognized technique. For example, the progression or onset of a neurodegenerative disorder can be slowed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

The period of time over which the progression or onset of a neurodegenerative disorder is measured can be for example, one or more months or one or more years, e.g., about 6 months, about 1 year, about 18 months, about 2 years, about 36 months, about 3, about 4, about 5, about 6, about 7, about 8, about 9, about 10, about 11, about 12, about 13, about 14, about 15, about 16, about 17, about 18, about 19, or about 20 years, or any amount of months or years in between the values of about 6 months to about 20 years or more.

In another embodiment of the invention, a neurodegenerative disorder may be positively impacted by administration of an intranasal low dose aminosterol composition of the invention. A “positive impact” includes for example slowing advancement of the condition, improving symptoms, etc.

C. Treatment and/or Prevention of Sleep Disorders

In some embodiments, the present disclosure provides methods of treating or preventing a sleep disorder or sleep disturbance in a subject comprising administering to the subject an intranasal low dose aminosterol composition disclosed herein.

The intranasal low dose aminosterol compositions of the invention may also prevent the onset of neurodegeneration. For example, subjects with conditions such as insomnia and sleeping disorders (detailed more specifically below), all have a much higher incidence of neurodegenerative disease. Thus, treating patient populations with sleep disorders may result in delaying or preventing the onset of neurodegenerative disease.

1. Overview Regarding Sleep Disorders

Normal sleep is critically important for the proper functioning of many organ systems, the most important of which is the brain. Disturbances in normal sleep patterns are closely associated with the normal aging process, with the development of cognitive impairment, with impaired memory deposition and consolidation and with the occurrence of neurodevelopmental, neuroaffective and neurodegenerative disorders. The alternating pattern of sleep and wakefulness occurring every 24 hours is known as the circadian rhythm. The rhythm is set by the “zeitgeber” (time setter), an entity known as the suprachiasmatic nucleus (SCN) and located in the hypothalamus. The SCN is normally “entrained” or synchronized by the external light-dark cycle. This relationship between external light and dark and the sleep wake cycle synchronized to it by the SCN can be over ridden during periods of hunger by neural signals emanating in the gut and relayed to the hypothalamus. The circadian sleep-wake cycle can also shift in response to changes in external light-dark cycles, such as the desynchronization that occurs during travel from one time zone to another (jet-lag). Under such circumstances, a progressive adjustment occurs until the SCN is resynchronized with the external light-dark cycle. A similar “phase-shift” and adjustment occurs in night-shift workers.

Under normal circumstances, the properly functioning SCN, synchronized to the external light-dark cycle and to neural signals emanating from the enteric nervous system, will regulate the sleep-wake cycle by sending neural and chemical signals to the surrounding structures and to portions of the brain stem involved in sleep and wakefulness. An individual with a properly functioning hypothalamus and brain stem will go to bed and fall asleep within minutes, remain asleep throughout the night, wake up in the morning and remain awake and alert throughout the day. During the night, the asleep individual will experience several cycles of sleep, beginning with light sleep, progressing through rapid eye movement sleep (REM-sleep) to deep sleep and back. Each complete sleep period lasts about 90 minutes. Periods of REM-sleep are closely associated with dreaming. During REM-sleep, neural signals emanating from certain parts of the brain stem ensure that skeletal muscles become “atonic” or are paralyzed, such that the individual can't “act out” their dreams.

Certain diseases and conditions may impair the normal functioning of the “zeitgebber” or circadian clock. These conditions may be reversible, such as desynchronization resulting from jet-lag, night-shift work or hunger, conditions easily remedied by adaptation or food intake. In contrast, damage to the nerves carrying light-dark related information from the retina to the SCN (conditions which may lead to blindness), or damage to the enteric nerves and neural structures which relay messages from the intestine to the SCN (conditions which may lead to neurodegenerative disorders) can cause permanent dysfunction of the circadian rhythm and abnormal sleep behavior.

Dysfunction of the circadian rhythm manifests first and foremost by abnormal sleep patterns. Such abnormalities typically are mild at onset and worsen progressively over time. A common symptom of sleep disorder is a delay in the onset of sleep. This delay can be as long as several hours, and the individual may not be able to fall asleep until the early hours of the morning. Another common symptom is sleep fragmentation, meaning that the individual awakens several times during the course of the night. Once awakened, the individual may not be able to get back to sleep, and each awake fragment may last an hour or more, further reducing “total sleep time,” which is calculated by subtracting total time of the awake fragments from total time spent in bed. Total sleep time also diminishes with age, from about 14 to about 16 hours a day in newborns, to about 12 hours by one year of age, to about 7 to about 8 hours in young adults, progressively declining to about 5 to about 6 hours in elderly individuals. Total sleep time can be used to calculate an individual's “sleep age” and to compare it to their chronologic age. Significant discrepancies between sleep age and chronologic age are a reflection of the severity of the sleep disorder. “Sleep efficiency,” defined as the percentage of the time spent in bed asleep is another index that can be used to determine the severity of the sleep disorder. Sleep efficiency is said to be abnormal when the percentage is below 70%.

Individuals with severe sleep disorders also typically suffer from day-time sleepiness. This can manifest as day-time “napping” for an hour or two, to “dosing off” for a few minutes during a film or to “micro-sleep” episodes lasting seconds to minutes, and of which the individual may or may not be aware. Narcolepsy is a rare and extreme form of day-time sleepiness, with the sudden onset of sleep causing the individual to fall down. Another form of sleep disturbance involves periods of loud snoring alternating with periods of “sleep apnea” (arrested breathing), a condition known as “sleep-disordered breathing.” “REM-behavior disorder” or RBD, is yet another sleep disturbance which occurs as a result of dysfunctional neural communication between the enteric nervous system, structures responsible for sleep in the brain stem and the SCN. In individuals with RBD, neural signaling which causes the paralysis (atonia) of muscles under voluntary control is impaired or altogether absent. As a consequence, “acting-out” of dreams occurs. This can range at one end of the spectrum from an increase in muscle tone detectable by electromyography (EMG) and accompanied by small movements of the hands and feet during REM sleep, to violent thrashing of arms and legs, kicking or punching a bed partner, speaking out loud or screaming, at the other end of the spectrum. Episodes of RBD can occur several times a night or very infrequently, once every few months. They can also be clustered, several occurring within a week, followed by periods of normal sleep. Unless the condition can be treated with a medication that restores normal functioning of the circadian rhythm and improves sleep patterns, individuals with RBD progress to neurodegenerative disorders.

Sleep disorders are very commonly associated with dysfunction of the enteric nervous system (ENS). As a result of the dysfunction of the enteric nerves, motility is impaired and the individual typically suffers from constipation. Furthermore, as a consequence of the pathology in the ENS, neural signaling between the ENS, the brain stem and the SCN is impaired and normal sleep behavior is disturbed. Untreated, the pathologic process which begins in the ENS extends via the neural connections to the brain stem and hypothalamus. There is thus a need for a treatment which restores the proper function of the ENS, improving gut motility, overcoming constipation and which simultaneously improves neural signaling from ENS to brainstem and SCN, restoring the circadian rhythm and normal sleep behavior.

Sleep disorders are also associated with olfactory dysfunction. Many if not most individuals with sleep disorders have an impaired or absent sense of smell. As a consequence of the pathology in the olfactory system, neural signaling between the olfactory system and SCN is disturbed. As is the case for motility disorders emanating from the ENS, the pathologic process that begins in the olfactory system causing loss of smell spreads progressively to the basal forebrain, the hypothalamus and the brain stem, resulting in a sleep disorder. There is thus a need for a treatment which restores the proper function of the olfactory system, improving the sense of smell, and which simultaneously improves neural signaling from the olfactory system to the SCN, restoring the circadian rhythm and normal sleep behavior.

Sleep disorders are also closely associated with blunting of diurnal temperature oscillations. In a normally functioning individual, vasomotor changes regulated by the hypothalamus modify distal extremity temperature, allowing it to rise to 34-36° C. at night, to remain high during the night, then to fall to 28-30° C. in the early morning hours and to remain low throughout the day. The heat loss that ensues from the rise in extremity temperature leads to a corresponding but somewhat delayed fall in core body temperature (CBT). About one hour after hand temperature peaks, CBT is reduced by 1° C. Sleep onset is closely linked to this nadir in CBT. The reverse happens in the morning, with CBT rising by a degree about an hour after hand temperature reaches its nadir and the individual awakens. As long as hand temperature remains low, the individual stays awake. Hand temperature is thus a close indicator of the sleep-wake state and can be used as a surrogate measure to determine when sleep occurs. In individuals with sleep disorders, diurnal oscillations in skin temperature are blunted or even absent. Hand temperature doesn't rise in the evening hours and sleep onset is delayed. During the night, it may fall below 34° C. and the individual then awakens and remains awake until it rises again. Repeated falls in hand temperature are paralleled by repeated awakenings or fragmented sleep. Contrarily, during the day, hand temperature fails to remain low, rising above a threshold intermittently. The individual with a sleep disorder correspondingly experiences day-time sleepiness. Fluctuation in skin temperature is thus a reliable marker of circadian physiology and it has been validated as a surrogate to polysomnography to assess disturbances of sleep. It can be monitored non-invasively, continuously, in a home setting and at very low cost using validated temperature sensors.

The neurologic structures involved in the induction and maintenance of sleep and in arousal and maintenance of wakefulness are concentrated in the hypothalamus and brain stem. While the SCN is responsible for setting the circadian rhythm, the pre-optic nuclei are responsible for sleep onset and sleep maintenance (MnPO and VLPO respectively). The pedunculopontine nucleus (PPN) in the brain stem is responsible for oscillations between REM and non-REM sleep and the raphe nuclei and the reticular activating system (RAS) regulate arousal. All these structures receive input from the SCN and the pre-optic nuclei in the hypothalamus and basal forebrain. The firing rate of each of these structures is state-specific. Structures involved in wakefulness such as the RAS fire fastest during wakefulness, slow down during non-REM sleep, and nearly stop firing during REM sleep. The suppression of motor activity during normal REM sleep (atonia) is the result of multiple interacting inhibitory pathways emanating from a region known as subcoeruleus and terminating on spinal motor neurons. Changes in neuronal firing rates in relevant centers in turn influence the secretion of hypothalamic proteins such as hypocretins that also affect sleep-wake cycles and muscle tone. Many or all of these structures are dysfunctional in individuals with sleep disorders.

Sleep disorders are not only a consequence of pathologic processes that begin in the ENS or in the olfactory system but they may actively contribute to the progression of such pathologic processes. The pathologic protein (a-beta) associated with plaques found in Alzheimer's disease (AD) for example, may be cleared from the brain during normal sleep, and sleep disorders may interfere with its nocturnal clearance. Even small alterations in brain a-beta levels could significantly increase plaque pathology over a long timeframe, setting in motion a cycle in which progression of a-beta pathology further impairs the sleep disorder. The same vicious cycle may be true in Parkinson's disease (PD) in which the concentration of the pathologic, pro-inflammatory protein alpha-synuclein rises in the presence of a sleep disorder. Individuals with AD or PD and a sleep disorder are much more likely to develop dementia than individuals with the same disease but without the sleep disorder. Sleep disorders tend to occur long before a diagnosis of PD or AD, so the possibility exists that treating a sleep disorder before the neurodegenerative pathology develops might actually prevent or at the very least slow the development of the disease. Sleep disturbances are also common in individuals with schizophrenia and the worsening of the sleep disturbance can predict the occurrence of psychotic episodes.

There are currently no satisfactory treatments for sleep disorders associated with circadian dysfunction. The first line of treatment, clonazepam, is useful in inducing sleep in individuals with sleep disorders, and it may also alleviate symptoms of RBD but it does nothing to restore circadian rhythmicity. Its effect tends to wear off with continued use and because of its long duration of action, it may also worsen cognitive function and motor performance in the morning. It may also worsen sleep apnea because of its tendency to suppress brain stem neuronal activity. Polysomnographic features of RBD are not usually suppressed by clonazepam. Melatonin, a hormone secreted by the pineal gland, may be preferable to clonazepam because it can help restore circadian rhythmicity and can suppress RBD without the side-effects associated with clonazepam. It is also preferable to clonazepam because it is less sedating. However, melatonin does little to induce sleep in many of these patients. Recently, melatonin receptor agonists ramelteon and tasimelteon were approved for the treatment of circadian disturbance in the blind. Other drugs which have been tried in RBD with variable success include benzodiazepines, pramipexole, donepezil, levodopa, carbamazepine, triazolam, sodium oxybate, quetiapine and Nuplazid. The ideal medication for sleep disorders in this patient population would aim to improve sleep quality as well as normalizing circadian rhythmicity by directly targeting the circadian clock.

2. Examples of Sleep Disorders

Sleep disorders and/or sleep disturbances include but are not limited to REM-behavior disorders, disturbances in the Circadian rhythm, delayed sleep onset, sleep fragmentation, and hallucinations. Other sleep disorders or disturbances that can be treated and/or prevented according to the disclosed methods include but are not limited to hypersomnia (i.e., daytime sleepiness), parasomnias (such as nightmares, night terrors, sleepwalking, and confusional arousals), periodic limb movement disorders (such as Restless Leg Syndrome), jet lag, narcolepsy, advanced sleep phase disorder, non-24 hour sleep-wake syndrome.

Jet lag is a traveler's sleep condition that affects the body's internal clock by causing sleep disruptions in a new time zone. The severity of jet lag depends on many factors-including age, quantity of time zones, and the direction of travel-causing the sufferer to become fatigued, nauseated, headachy, and unable to fall to sleep. Advanced sleep phase disorder (or ASPD) occurs when the biological clock sets to rise earlier than it should—for instance, the sufferer may feel the need to sleep at 7 pm and wake up at 3 am. ASPD most commonly affects seniors and is often linked to seasonal affective disorder (or SAD). Narcolepsy is one of the most dangerous sleep disorders. It's rare, affecting only roughly 100,000 Americans. The condition itself causes a dysfunction in the brain mechanisms that manage sleeping and waking-causing a person to instantly fall asleep while conversing, walking, driving, climbing stairs, working, etc. Most narcoleptics are extremely fatigued during the daytime hours, and suffer from hallucinations, muscle deterioration, sleep paralysis, and fainting. Subjects going to sleep earlier and earlier or waking up later and later, may have non-24-hour-sleep-wake syndrome, a condition that sets their biological clock to 25 hours or longer. This condition is often linked to blind individuals due to the absence of waking and sleeping light cues. Restless Leg Syndrome (or RLS) causes the lower legs to burn, ache, itch, twitch, and tingle upon falling sleep. It disrupts sleep mostly in middle-aged sufferers and is associated with a family history of RLS. Insomnia refers to a condition where subjects have a difficult time falling or staying asleep.

Approximately 70 million Americans suffer from one sleep disorder or another. Sleep disorders are characterized by any condition that prevents a person from getting restful sleep for a desirable period of time. The dangerous part isn't the actual sleep loss, but the dysfunction it causes during the waking hour when subjects are operating motor vehicles, work-associated machinery, and so forth.

Sleep is increasingly recognized as important to public health, with sleep insufficiency linked to motor vehicle crashes, industrial disasters, and medical and other occupational errors. Unintentionally falling asleep, nodding off while driving, and having difficulty performing daily tasks because of sleepiness all may contribute to these hazardous outcomes. Persons experiencing sleep insufficiency are also more likely to suffer from chronic diseases such as hypertension, diabetes, depression, and obesity, as well as from cancer, increased mortality, and reduced quality of life and productivity. Sleep insufficiency may be caused by broad scale societal factors such as round-the-clock access to technology and work schedules, but sleep disorders such as insomnia or obstructive sleep apnea also play an important role. An estimated 50-70 million US adults have a sleep or wakefulness disorder.

3. Patient Populations

In another embodiment of the invention, encompassed is a method of treating or preventing a sleep disorder or sleep disturbance in a subject suffering from a neurodegenerative condition of the CNS, comprising administering to the subject an intranasal low dose aminosterol composition according to the invention. In other embodiments of the invention, the neurodegenerative condition of the CNS is such as Parkinson's disease (PD), supranuclear palsy, multi-system atrophy, Parkinsonism, Alzheimer's disease, frontotemporal dementia, amyotrophic lateral sclerosis (ALS), Huntington's Disease, schizophrenia, Friedreich's ataxia, Multiple sclerosis (MS), Lewy Body dementia or disease, spinal muscular atrophy, frontotemporal dementia, progressive nuclear palsy, Guadeloupian Parkinsonism, spinocerebellar ataxia, or vascular dementia. The method preferably results in the subject obtaining a restful sleep period. A “restful sleep period” is defined as a sleep period uninterrupted by wakefulness. Alternatively, a “restful sleep period” can be defined by the recommended or appropriate amount of sleep for the subject's age category, e.g., infants 0-3 months=11-19 hours; infants 4-11 months=12-18 hours; toddlers 1-2 years=9-16 hours; preschoolers 3-5 years=10-14 hours; school-aged children 6-13 years=7-12 hours; teenagers 14-17 years=7-11 hours; young adults 18-25 years=6-11 hours; adults 26-64 years=6-10 hours; and older adults ≥65 years=5-9 hours. Thus, for treating a sleep disorder or sleep disturbance in a subject suffering from a neurodegenerative condition of the CNS, the treatment can result in a restful sleep period of at least about 5, about 6, about 7, about 8, about 9, about 10, about 11, or about 12 hours.

In another embodiment of the invention, encompassed is a method of treating or preventing a sleep disorder or sleep disturbance in a subject suffering from degenerative processes associated with aging, comprising administering to the subject an intranasal low dose aminosterol pharmaceutically composition according to the invention. The method preferably results in the subject obtaining a restful sleep period. For example, the restful sleep period can comprise at least about 5, about 6, about 7, about 8, about 9, about 10, about 11, or about 12 hours.

How much sleep is needed by a subject varies between individuals but generally changes with age. The National Institutes of Health suggests that school-age children need at least 10 hours of sleep daily, teens need 9-10 hours, and adults need 7-8 hours. According to data from the National Health Interview Survey, nearly 30% of adults reported an average of ≤6 hours of sleep per day in 2005-2007. Further, in 2009, only 31% of high school students reported getting at least 8 hours of sleep on an average school night. Similar recommendations are provided by the National Sleep Foundation (https://sleepfoundation.org/press-release/national-sleep-foundation-recommends-new-sleep-times/page/0/1): Table

TABLE 1

| | | May be | |
|---|---|---|---|
| Age | Recommended | appropriate | Not recommended |
| Newborns | 14 to 17 hours | 11 to 13 hours | Less than 11 hours |
| 0-3 months | | 18 to 19 hours | More than 19 hours |
| Infants | 12 to 15 hours | 10 to 11 hours | Less than 10 hours |
| 4-11 months | | 16 to 18 hours | More than 18 hours |
| Toddlers | 11 to 14 hours | 9 to 10 hours | Less than 9 hours |
| 1-2 years | | 15 to 16 hours | More than 16 hours |
| Preschoolers | 10 to 13 hours | 8 to 9 hours | Less than 8 hours |
| 3-5 years | | 14 hours | More than 14 hours |
| School-aged | 9 to 11 hours | 7 to 8 hours | Less than 7 hours |
| Children | | 12 hours | More than 12 hours |
| 6-13 years | | | |
| Teenagers | 8 to 10 hours | 7 hours | Less than 7 hours |
| 14-17 years | | 11 hours | More than 11 hours |
| Young Adults | 7 to 9 hours | 6 hours | Less than 6 hours |
| 18-25 years | | 10 to 11 hours | More than 11 hours |
| Adults | 7 to 9 hours | 6 hours | Less than 6 hours |
| 26-64 years | | 10 hours | More than 10 hours |
| Older Adults | 7 to 8 hours | 5 to 6 hours | Less than 5 hours |
| ≥65 years | | 9 hours | More than 9 hours |

In one aspect of the invention, encompassed is a method of treating or preventing a sleep disorder or sleep disturbance in a subject, comprising administering to the subject an intranasal low dose aminosterol composition according to the invention, wherein the method results in the subject obtaining a restful sleep period. A “restful sleep period” can also be defined by the recommended or appropriate amount of sleep for the subject's age category, e.g., infants 0-3 months=11-19 hours; infants 4-11 months=12-18 hours; toddlers 1-2 years=9-16 hours; preschoolers 3-5 years=10-14 hours; school-aged children 6-13 years=7-12 hours; teenagers 14-17 years=7-11 hours; young adults 18-25 years=6-11 hours; adults 26-64 years=6-10 hours; and older adults >65 years=5-9 hours.

There are several different scientifically acceptable ways to measure a sleep period uninterrupted by wakefulness. First, electrodes attached to the head of a subject can measure electrical activity in the brain by electroencephalography (EEG). This measure is used because the EEG signals associated with being awake are different from those found during sleep. Second, muscle activity can be measured using electromyography (EMG), because muscle tone also differs between wakefulness and sleep. Third, eye movements during sleep can be measured using electro-oculography (EOG). This is a very specific measurement that helps to identify Rapid Eye Movement or REM sleep. Any of these methods, or a combination thereof, can be used to determine if a subject obtains a restful sleep period following administration of at least one aminosterol to the subject.

In another aspect of the invention, encompassed is a method of treating or preventing a sleep disorder or sleep disturbance in an infant subject, comprising administering to the infant subject an intranasal low dose aminosterol composition according to the invention. The method preferably results in the subject obtaining a restful sleep period of at least about 5, about 6, about 7, about 8, about 9, about 10, about 11, about 12, about 13, about 14, about 15, or about 16 hours. “Infant” subjects can be anywhere from 0 to 12 months of age.

In another aspect of the invention, encompassed is a method of treating or preventing a sleep disorder or sleep disturbance in a toddler subject, comprising administering to the subject an intranasal low dose aminosterol composition according to the invention. The method preferably results in the subject obtaining a restful sleep period of at least about 5, about 6, about 7, about 8, about 9, about 10, about 11, about 12, about 13, about 14, about 15, or about 16 hours. “Toddler” subjects can be from 1-2 years, up to less than 3 years.

In another aspect of the invention, encompassed is a method of treating or preventing a sleep disorder or sleep disturbance in a preschooler subject, comprising administering to the infant subject an intranasal low dose aminosterol composition according to the invention. The method preferably results in the subject obtaining a restful sleep period of at least about 5, about 6, about 7, about 8, about 9, about 10, about 11, about 12, about 13, or about 14 hours. “Preschooler” subjects can be from 3-5 years, up to less than 6 years.

In another aspect of the invention, encompassed is a method of treating or preventing a sleep disorder or sleep disturbance in a school-aged children, administering to the subject an intranasal low dose aminosterol composition according to the invention. The method preferably results in the subject obtaining a restful sleep period of at least about 5, about 6, about 7, about 8, about 9, about 10, about 11, or about 12. “School-aged children” subjects can be from 6-13 years, up to less than 14 years.

In another aspect of the invention, encompassed is a method of treating or preventing a sleep disorder or sleep disturbance in a teenage subject, administering to the subject an intranasal low dose aminosterol composition according to the invention. The method preferably results in the subject obtaining a restful sleep period of at least about 5, about 6, about 7, about 8, about 9, about 10, or about 11 hours. “Teenage” subjects can be from 14-17 years, up to less than 18 years.

In another aspect of the invention, encompassed is a method of treating or preventing a sleep disorder or sleep disturbance in a young adult subject, administering to the subject an intranasal low dose aminosterol composition according to the invention. The method preferably results in the subject obtaining a restful sleep period of at least about 5, about 6, about 7, about 8, about 9, about 10, or about 11 hours. “Young adult” subjects can be about 18-25 years, up to less than 26 years.

In another aspect of the invention, encompassed is a method of treating or preventing a sleep disorder or sleep disturbance in an adult subject, administering to the subject an intranasal low dose aminosterol composition according to the invention. The method preferably results in the subject obtaining a restful sleep period of at least about 5, about 6, about 7, about 8, about 9, or about 10 hours. “Adult” subjects can be about 26-64 years, up to less than 65 years.

In another aspect of the invention, encompassed is a method of treating or preventing a sleep disorder or sleep disturbance in an elderly or older adult subject, administering to the subject an intranasal low dose aminosterol composition according to the invention. The method preferably results in the subject obtaining a restful sleep period of at least about 5, about 6, about 7, about 8, or about 9 hours. “Elderly” or “older adult” subjects can be 65 or more years of age.

D. Treatment and/or Prevention of Hallucinations

In one embodiment, the invention is directed to a method of treating or preventing hallucinations in a subject comprising administering to the subject an intranasal low dose aminosterol pharmaceutical composition according to the invention. The hallucination can comprise, for example, a visual, auditory, tactile, gustatory or olfactory hallucination.

In an exemplary embodiment, the hallucination is the result of a psychiatric or neurological disorder. The intranasal low dose aminosterol composition can, for example, reverse the dysfunction of the psychiatric or neurological disorder and treat the hallucination. The psychiatric disorder can be, for example, selected from the group consisting of Bipolar disorder, Borderline personality disorder, Depression (mixed), Dissociative identity disorder, Generalized anxiety disorder, Major depression, Obsessive compulsive disorder, Post-traumatic stress disorder, Psychosis (NOS), Schizoaffective disorder, and Schizophrenia.

In another embodiment, the hallucination can be the result of a neurological disorder. The neurological disorder can be, for example, the result of (a) a brain tumor, (b) a sleep disorder such as narcolepsy, or (c) a focal brain lesion, such as occipital lobe lesions or temporal lobe lesions. In an exemplary embodiment, the temporal lobe lesion can be lesions of the uncinate gyrus, cerebral peduncles, or substantia nigra. The neurological disorder can be, for example, the result of (d) a diffuse involvement of the cerebral cortex, such as that caused by a viral infectious disease.

The diffuse involvement of the cerebral cortex can be a result of a cerebral vasculitis condition, and the viral infectious disease can be, for example, acute metabolic encephalopathies, encephalitis, or meningitis. The cerebral vasculitis condition can be caused by an autoimmune disorder, a bacterial or viral infection, or a systemic vasculitis. The autoimmune disorder can be, for example, Systemic Lupus Erythematosus (SLE).

In yet another embodiment, the hallucination can be the result of a neurodegenerative disorder. For example, the neurodegenerative disorder can be, for example, such as Parkinson's disease (PD), supranuclear palsy, multi-system atrophy, Parkinsonism, Alzheimer's disease, frontotemporal dementia, amyotrophic lateral sclerosis (ALS), Huntington's Disease, schizophrenia, Friedreich's ataxia, Multiple sclerosis (MS), Lewy Body dementia or disease, spinal muscular atrophy, frontotemporal dementia, progressive nuclear palsy, Guadeloupian Parkinsonism, spinocerebellar ataxia, or vascular dementia. In a preferred embodiment, the intranasal low dose aminosterol compositions of the invention reverse the dysfunction of the neurodegenerative disorder and treat the hallucination.

In another embodiment, the hallucination is caused by a sensory loss. The sensory loss can be, for example, visual, auditory, gustatory, tactile, or olfactory. In a preferred embodiment, the intranasal low dose aminosterol compositions of the invention reverse the dysfunction of the sensory loss and treat the hallucination. In a preferred embodiment, the intranasal low dose aminosterol compositions of the invention reverse the dysfunction of the enteric nervous system and treats the hallucination.

The methods of using an intranasal low dose aminosterol composition according to the invention to treat and/or prevent hallucinations preferably result in a decrease in hallucinations. The decrease can be, for example, a reduction in occurrences of hallucinations by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The methods of the invention may also result in the subject being hallucination-free.

A hallucination is a sensory impression or perception of an object or event, in any of the 5 senses (sight, touch, sound, smell, or taste) that has no basis in external stimulation. Hallucinations can have debilitating impact on the subject's health and life by causing harm to self or others, by making it difficult for the subject to function normally in everyday situations, and by causing sleep disruption. Examples of hallucinations include “seeing” someone not there (visual hallucination), “hearing” a voice not heard by others (auditory hallucination), “feeling” something crawling up your leg (tactile hallucination), “smelling” (olfactory), and “tasting” (gustatory). Other examples of hallucination types include hypnagogic hallucination (a vivid, dreamlike hallucination occurring at sleep onset), hypnopompic hallucination (a vivid, dreamlike hallucination occurring on awakening), kinesthetic hallucination (a hallucination involving the sense of bodily movement), and somatic hallucination a hallucination involving the perception of a physical experience occurring within the body.

Hallucinations can be a result of psychiatric conditions. Hallucinations, especially auditory hallucinations, are characteristic of certain psychiatric conditions such as schizophrenia, occurring in up to 70-80% of subjects. They also occur in 30-50% of individuals with borderline personality disorder. Auditory hallucinations can take control of actions or behavior and elicit violent defensive behavior or alternatively lead to self-harming behavior. They can also occur in post-partum psychosis. Auditory hallucinations can less commonly occur in severely depressed patients or even in mania. Substance abuse can also be associated with visual hallucinations. Alcohol intoxication or withdrawal, post-traumatic stress disorder (PTSD) and bereavement can also be associated with visual hallucinations.

Hallucinations can be a result of neurological disorders. In one embodiment the neurological disorder is a brain tumor. In some embodiments, the “focal brain lesions.” Formed and unformed visual hallucinations can occur in the presence of temporal and occipital lobe lesions. Occipital lobe lesions typically produce simple geometric patterns or “strings of circles like a bunch of grapes” or stars which can follow the gaze (palinopsia), whereas temporal lobe lesions are associated with complex, formed hallucinations. Temporal lobe lesions and especially lesions of the uncinate gyrus are typically associated with olfactory and gustatory hallucinations. Lesions of the cerebral peduncles and substantia nigra are associated with “peduncular hallucinosis” or colorful vivid images. In some embodiments, the hallucinations are a result of diffuse involvement of the cerebral cortex. In some embodiments, of diffuse involvement Acute metabolic encephalopathies and encephalitis caused by viral infections or diseases associated with a cerebral vasculitis such as Systemic Lupus Erythematosus (SLE) can cause visual hallucinations.

E. Treatment and/or Prevention of Depression

The present invention also encompasses methods of treating and/or preventing depression comprising administering an intranasal low dose aminosterol composition according to the invention.

Clinical depression is a mood disorder that is characterized by a sad, blue mood that goes above and beyond normal sadness or grief. Major depression is an episode of sadness or apathy along with other symptoms that lasts at least two consecutive weeks and is severe enough to interrupt daily activities. Depressive disorders feature not only negative thoughts, moods, and behaviors but also specific changes in bodily functions (like, eating, sleeping, energy and sexual activity, as well as potentially developing aches or pains). One in 10 people will have a depression in their lifetime. Doctors clinically diagnose depression; there is no laboratory test or X-ray for depression.

Increasingly sophisticated forms of brain imaging, such as positron emission tomography (PET), single-photon emission computed tomography (SPECT), and functional magnetic resonance imaging (fMRI), permit a much closer look at the working brain than was possible in the past. An fMRI scan, for example, can track changes that take place when a region of the brain responds during various tasks. A PET or SPECT scan can map the brain by measuring the distribution and density of neurotransmitter receptors in certain areas. Use of this technology has led to a better understanding of which brain regions regulate mood and how other functions, such as memory, may be affected by depression. Areas that play a significant role in depression are the amygdala, the thalamus, and the hippocampus.

Research shows that the hippocampus is smaller in some depressed people. For example, in one fMRI study published in The Journal of Neuroscience, investigators studied 24 women who had a history of depression. On average, the hippocampus was 9% to 13% smaller in depressed women as compared with those who were not depressed. The more bouts of depression a woman had, the smaller the hippocampus. Stress, which plays a role in depression, may be a key factor, since experts believe stress can suppress the production of new neurons (nerve cells) in the hippocampus.

Researchers are exploring possible links between sluggish production of new neurons in the hippocampus and low moods. An interesting fact about antidepressants supports this theory. These medications immediately boost the concentration of chemical messengers in the brain (neurotransmitters). Yet people typically don't begin to feel better for several weeks or longer. Experts have long wondered why, if depression were primarily the result of low levels of neurotransmitters, people don't feel better as soon as levels of neurotransmitters increase. The answer may be that mood only improves as nerves grow and form new connections, a process that takes weeks. In fact, animal studies have shown that antidepressants do spur the growth and enhanced branching of nerve cells in the hippocampus. So, the theory holds, the real value of these medications may be in generating new neurons (a process called neurogenesis), strengthening nerve cell connections, and improving the exchange of information between nerve circuits.

Thus, in one embodiment of the invention, encompassed are methods of treating and/or preventing depression comprising administering an intranasal low dose aminosterol composition according to the invention. It is theorized that the intranasal compositions of the invention trigger neurogenesis, which functions to combat depression.

In a preferred embodiment, the methods of the invention produce an improvement in a subject's clinical depression. An improvement in a subject's depression can be measured using any clinically-recognized measurement. For example, improvement can be measured using a depression rating scale. In one embodiment of the invention, following treatment a subject experiences an about 5, about 10, about 15, about 20, about 25, about 30, about 35, about 40, about 45, about 50, about 55, about 60, about 65, about 70, about 75, about 80, about 85, about 90, about 95 or an about 100% improvement.

F. Treatment and/or Prevention of Autism

In another embodiment of the invention, encompassed are methods of treating and/or preventing autism comprising administering to a subject in need an intranasal low dose aminosterol composition according to the invention.

Autism, or autism spectrum disorder, refers to a range of conditions characterized by challenges with social skills, repetitive behaviors, speech and nonverbal communication, as well as by unique strengths and differences. There are many types of autism, caused by different combinations of genetic and environmental influences.

Autism's most-obvious signs tend to appear between 2 and 3 years of age. In some cases, it can be diagnosed as early as 18 months. Some developmental delays associated with autism can be identified and addressed even earlier.

The Centers for Disease Control and Prevention (CDC) estimates autism's prevalence as 1 in 59 children in the United States. This includes 1 in 37 boys and 1 in 151 girls. Around one third of people with autism remain nonverbal. Around one third of people with autism have an intellectual disability. Certain medical and mental health issues frequently accompany autism. They include gastrointestinal (GI) disorders, seizures, sleep disturbances, attention deficit and hyperactivity disorder (ADHD), anxiety and phobias.

Experts are still uncertain about of all the causes of autism. In all likelihood, there are multiple causes. It appears that a number of different circumstances, including environmental, biologic, and genetic factors, set the stage for autism and make a child more likely to have the disorder. It is likely that genetics play a large factor in the development of autism. Identical twins are more likely to both be affected than twins who are fraternal (not genetically identical). In a family with one autistic child, the chance of having another child with autism is about 5 percent—or one in 20—which is much higher than in the normal population. Research also has found that some emotional disorders (such as manic depression) occur more often in families of a child with autism.

At least one group of researchers has found a link between an abnormal gene and autism. The gene may be just one of three to five or more genes that interact in some way to cause the condition. Scientists suspect that a faulty gene or genes might make a person more likely to develop autism when there are also other factors present, such as a chemical imbalance, viruses or chemicals, or a lack of oxygen at birth.

Other potential causes of autism are environmental toxins, including pesticides and heavy metals such as mercury. Heavy metals are certainly more commonly encountered in the environment now than they were in the past. It may be that people with autism or those at higher risk for developing it are more sensitive than others to these toxins.

A recent brain-tissue study suggests that children affected by autism have a surplus of synapses, or connections between brain cells. The excess is due to a slowdown in the normal pruning process that occurs during brain development. During normal brain development, a burst of synapse formation occurs in infancy. This is particularly pronounced in the cortex, which is central to thought and processing information from the senses. But by late adolescence, pruning eliminates about half of these cortical synapses. In addition, many genes linked to autism are known to affect the development or function of brain synapses. The study also found that The brain cells from the individuals with autism were filled with damaged parts and deficient in signs of a normal breakdown pathway called “autophagy.” Tang et al., “Loss of mTOR-Dependent Macroautophagy Causes Autistic-like Synaptic Pruning Deficits,” Neuron, 83(5):1131-1143 (2014).

Thus, one embodiment of the invention is directed to methods of treating autism comprising administering an intranasal low dose aminosterol composition according to the invention. It is theorized that the IN low dose aminosterol compositions of the invention trigger neurogenesis which results in addressing the damaged brain synapses. In one embodiment, treatment results in improvement in one or more characteristics of autism. Such characteristics can be, for example, communication skills, social interaction, sensory sensitivity, and behavior.

For example, the methods of the invention may show an improvement in one or more characteristics of autism, such as behavior, communication, mood, etc., as measured by a medically recognized scale. The improvement may be, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

G. Schizophrenia

In another embodiment of the invention, encompassed are methods of treating schizophrenia comprising administering to a subject in need an intranasal low dose aminosterol composition according to the invention.

Schizophrenia is a mental disorder characterized by abnormal social behavior and failure to understand reality. Common symptoms include false beliefs, unclear or confused thinking, hearing voices that others do not, reduced social engagement and emotional expression, and a lack of motivation. People with schizophrenia often have additional mental health problems such as anxiety, depressive, or substance-use disorders. Symptoms typically come on gradually, begin in young adulthood, and last a long time. Schizophrenia affects roughly 3.5 million people, or about one percent of the U.S. population, according to the National Institutes of Health. Globally, some 24 million are affected, according to the World Health Organization.

Schizophrenia is a chronic progressive disorder that has at its origin structural brain changes in both white and gray matter. It is likely that these changes begin prior to the onset of clinical symptoms in cortical regions, particularly those concerned with language processing. Later, they can be detected by progressive ventricular enlargement. Current magnetic resonance imaging (MRI) technology can provide a valuable tool for detecting early changes in cortical atrophy and anomalous language processing, which may be predictive of who will develop schizophrenia.

A 2013 study of schizophrenia patients documented brain changes seen in MRI scans from more than 200 patients beginning with their first episode and continuing with scans at regular intervals for up to 15 years. The scans showed that people at their first episode had less brain tissue than healthy individuals. The findings suggest that those who have schizophrenia are being affected by something before they show outward signs of the disease.

The mainstay of treatment is antipsychotic medication, along with counselling, job training and social rehabilitation. However, the 2013 study found that in general, the higher the anti-psychotic medication doses, the greater the loss of brain tissue.

About 0.3-0.7% of people are affected by schizophrenia during their lifetimes. In 2013 there were an estimated 23.6 million cases globally. Males are more often affected, and on average experience more severe symptoms. About 20% of people do well and a few recover completely. About 50% have lifelong impairment. Social problems, such as long-term unemployment, poverty and homelessness are common. The average life expectancy of people with the disorder is ten to twenty-five years less than for the general population. This is the result of increased physical health problems and a higher suicide rate (about 5%). In 2015 an estimated 17,000 people worldwide died from behavior related to, or caused by, schizophrenia.

It is theorized that administration of intranasal low dose aminosterol compositions of the invention to a schizophrenia patient results in stimulation of neurogenesis, which has a positive impact on the loss of brain tissue characteristic of schizophrenia subjects.

In one embodiment of the invention, administration of intranasal low dose aminosterol compositions of the invention to a schizophrenia patient results in improvement of one or more symptoms as determined by a clinically recognized psychiatric symptom rating scale. Examples of such rating scales include for example, the Positive and Negative Syndrome Scale (PANSS), the Psychotic Symptom Rating Scales (PSYRATS), the Quality of Life Scale (QLS), the Schizophrenia Cognition Rating Scale (SCoRS), the Drug Attitude Inventory (DAI), and the Abnormal Involuntary Movement Scale (AIMS).

In another embodiment, administration of intranasal low dose aminosterol compositions of the invention to a schizophrenia patient results in improvement of one or more symptoms as determined by a clinically recognized psychiatric symptom rating scale, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

H. Treatment and/or Prevention of Inflammatory Conditions

In another embodiment, encompassed are methods of treating and/or preventing inflammatory diseases and conditions caused by excessive expression of neuronal alpha-synuclein comprising administering to a subject in need an intransal low dose aminosterol composition according to the invention.

Alpha-synuclein is a potent pro-inflammatory hormone. Inflammation can be blocked by either of two strategies. First, inflammation can be blocked by reducing the tissue concentration of alpha-synuclein by decreasing or stopping production of alpha-synuclein. Alternatively, inflammation can be blocked by interrupting the signaling between alpha-synuclein and inflammatory cells that express CD11b. The subject of the methods of the invention can be any mammal, including a human.

The inflammatory disease or condition caused by excessive expression of neuronal alpha synuclein can be a neurodegenerative disorder (NDD), such as an alpha-synucleinopathy. Exemplary alpha-synucleinopathies include, but are not limited to, Parkinson's disease, Lewy body dementia, multiple system atrophy, amytrophic lateral sclerosis, Huntington's chorea, multiple sclerosis or schizophrenia. In other embodiments, the inflammatory disease or condition caused by excessive expression of neuronal alpha synuclein can be an autoimmune disease, a chronic inflammatory disease, or an autoinflammatory disease. In other embodiments, the inflammatory disease or condition caused by excessive expression of neuronal alpha synuclein can be selected from the group consisting of asthma, chronic peptic ulcer, tuberculosis, chronic periodontitis, chronic sinusitis, chronic active hepatitis, psoriatic arthritis, gouty arthritis, acne vulgaris, osteoarthritis, rheumatoid arthritis, lupus, systemic lupus erythematosus, multiple sclerosis, ankylosing spondylitis, Crohn's disease, psoriasis, primary sclerosing cholangitis, ulcerative colitis, allergies, inflammatory bowel diseases, Celiac disease, Chronic prostatitis, diverticulitis, dermatomyositis, polymyositis, systemic sclerosis, glomerulonephritis, hidradenitis suppurativa, hypersensitivities, interstitial cystitis, otitis, pelvic inflammatory disease, reperfusion injury, rheumatic fever, sarcoidosis, transplant rejection, and vasculitis.

In some embodiments of the invention, patient populations particularly susceptible to excessive production or secretion of alpha-synuclein can benefit from the methods of the invention and are targeted for therapy, including for example preventative therapy. For example, a patient population having a mutated form of alpha-synuclein resulting in increased amounts of alpha-synuclein in tissues can be treated using the methods of the invention. Another example of a patient population susceptible for high levels of alpha-synuclein are patients having chronic inflammatory conditions or diseases.

The methods of the invention can result in a decrease in intensity of inflammation, blood levels of inflammatory markers, inflammatory markers in tissue, or number of inflammatory cells in tissue, or a combination thereof, as compared to a control or as compared to the qualitative or quantitative amount from the same patient or subject prior to treatment. For example, the decrease can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments of the invention, patient populations particularly susceptible to excessive production or secretion of alpha-synuclein can benefit from the methods of the invention and are targeted for therapy, including for example preventative therapy. For example, a patient population having a mutated form of alpha-synuclein resulting in increased amounts of alpha-synuclein in tissues can be treated using the methods of the invention. Another example of a patient population susceptible for high levels of alpha-synuclein are patients having chronic inflammatory conditions or diseases.

In addition, it follows from the present invention that an individual with an inflammatory condition appropriate for treatment or prophylaxis with the methods targeting alpha-synuclein described herein can be identified by determination of the tissue concentrations of alpha synuclein at sites of inflammation, with high levels of alpha-synuclein, as compared to a control or healthy subject, correlating with patients appropriate for treatment with a method of the invention.

In another method of the invention, encompassed is a method of identifying a subject with a condition amenable to treatment targeting alpha-synuclein CD11b interaction. The method comprises identifying a subject having an elevated concentration of alpha-synuclein present in a tissue, using either qualitative, quantitative, or semi-quantitative methods. For example, the method can comprise: (a) obtaining a tissue sample from a site of inflammation from the subject; and (b) qualitatively, quantitatively or semi-quantitatively determining the concentration of alpha synuclein within the tissue sample; wherein an elevated concentration of alpha-synuclein present in the tissue, as compared to a control or healthy subject, indicates that the subject is amenable to treatment targeting alpha-synuclein CD11b interaction. Other suitable methods of identifying subjects having an elevated concentration of alpha-synuclein present in a tissue are described herein and can also be used in the methods of the invention. For example, a subject amenable to treatment using methods of the invention can be identified by (a) measuring the intensity of inflammation over time; (b) measuring the amount of inflammatory markers over time; (c) measuring the amount of inflammatory markers in blood, plasma, or tissue over time, either qualitatively or quantitatively; (d) measuring the amount of one or more inflammatory marker cytokines in blood, plasma, or tissue over time, either qualitatively or quantitatively; (e) measuring the amount of one or more plasma markers of inflammation such as TNF, IL-8, or CRP in blood, plasma, or tissue over time, either qualitatively or quantitatively; or (f) measuring the amount of inflammatory cells in blood, plasma, or tissue over time, either qualitatively or quantitatively.

I. Treatment and/or Prevention of Blood Pressure Conditions

Another aspect of the disclosure is directed to a method of treating, preventing, and/or slowing the onset or progression of high blood pressure (HBP) and/or a related symptom in a subject in need comprising low dose intranasal administration to the subject a therapeutically effective amount of at least one aminosterol, or a salt or derivative thereof.

In yet another aspect of the disclosure, encompassed is a method of treating, preventing, and/or slowing the onset or progression of low blood pressure (LBP) and/or a related symptom, in a subject in need comprising intranasal administration to the subject a therapeutically effective amount of at least one aminosterol, or a salt or derivative thereof.

The methods can result in slowing, halting, or reversing progression or onset of HBP and/or a related symptom, or LBP and/or a related symptom, over a defined period of time following administration of the aminosterol or a salt or derivative thereof, as measured by a medically-recognized technique. In another aspect, the HBP and/or a related symptom, or LBP and/or a related symptom is positively impacted by the dose of the aminosterol or a salt or derivative thereof, as measured by a medically-recognized technique. The positive impact and/or progression of HBP and/or a related symptom, or LBP and/or a related symptom can be measured quantitatively or qualitatively by one or more medically-recognized techniques selected from the group consisting of sphygmomanometry, arterial penetration, palpitation, asuculatoration, oscillometry, continuous noninvasive arterial pressure (CNAP), pulse wave velocity, and ambulatory monitoring; and/or the progression or onset of HBP and/or a related symptom, or LBP and/or a related symptom can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique. The clinically recognized scale or tool can be selected from the group consisting of sphygmomanometry, arterial penetration, palpitation, asuculatoration, oscillometry, continuous noninvasive arterial pressure (CNAP), pulse wave velocity, and ambulatory monitoring. Each defined period of time can be independently selected from the group consisting of about 1 day to about 10 days, about 10 days to about 30 days, about 30 days to about 3 months, about 3 months to about 6 months, about 6 months to about 12 months, and greater than about 12 months.

Examples of HBP or LBP symptoms (or related symptoms) include but are not limited to (a) a systolic blood pressure (BP) ≥120 and a diastolic BP<80; (b) a systolic blood pressure (BP)≥130 or a diastolic BP≥80; (c) headache; (d) lightheadedness; (e) vertigo; (f) tinnitus; (g) altered vision; (h) fainting; (i) hypertensive retinopathy; (j) palpitations; (k) excess sweating; (l) a systolic blood pressure ≤80; (m) a diastolic blood pressure ≤50; (n) fatigue; (o) stiff neck and/or upper back; (p) dyspepsia; (q) dysuria; (r) seizure; (s) shortness of breath; (t) constipation; (u) hallucinations; (v) depression; (w) sleep disorder, sleep problem, and/or sleep disturbance; (x) cardiovascular disease; and (y) cognitive impairment.

J. Treatment and/or Prevention of Cardiac Conduction Defects

Also encompassed by the disclosure is a method of treating, preventing, and/or slowing the onset or progression of a cardiac conduction defect (CCD) and/or a related symptom in a subject in need comprising low dose nasal administration to the subject a therapeutically effective amount of at least one aminosterol or a salt or derivative thereof.

In one aspect, progression or onset of CCD or a related symptom is slowed, halted, or reversed over a defined period of time following administration of the aminosterol or a salt or derivative thereof, as measured by a medically-recognized technique; and/or the CCD or related symptom is positively impacted by the aminosterol or a salt or derivative thereof, as measured by a medically-recognized technique. The positive impact and/or progression of CCD or a related symptom can be measured quantitatively or qualitatively by one or more techniques selected from the group consisting of echocardiography, electrocardiography (ECG or EKG), magnetic resonance imaging (MRI), positron-emission tomography (PET); coronary catheterization, intravascular ultrasound, Holter monitoring, stress test, computed tomography angiography (CTA), and coronary CT calcium scan; and/or the progression or onset of CCD or a related symptom can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In another aspect, nasal administration of the aminosterol or a salt or derivative thereof reverses dysfunction caused by the CCD and treats, prevents, improves, and/or resolves the symptom being evaluated. Optionally, the improvement or resolution of the CCD or related symptom can be measured using a clinically recognized scale or tool. In yet another aspect, the improvement in the CCD and/or related symptom is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale.

The CCD related symptom can be, for example, of a QT interval (QTc) ≥440 ms; syncope; presence of delta wave in electrocardiogram (EKG); pseudo-right bundle branch block in EKG; ST elevations in V1-V3 in EKG; a QRS complex >100 ms in EKG; PR interval <120 ms in EKG; heart rate above 100 beats per minute (BPM); heart rate below 60 BPM; PR interval >200 ms in EKG; QRS not following a P wave in EKG; no repeating relation between P wave and QRS complex in EKG; differing atrial and ventricular rates; QS or rS complex in lead V1 in EKG; notched (‘M’-shaped) R wave in lead V6; T wave discordance in EKG; left axis deviation between −45° and −60° in EKG; qR pattern (small q, tall R) in the lateral limb leads I and aVL in EKG; rS pattern (small r, deep S) in the inferior leads II, III, and aVF in EKG; delayed intrinsicoid deflection in lead aVL (>0.045 s) in EKG; frontal plane axis between 900 and 180° in EKG; rS pattern in leads I and aVL in EKG; qR pattern in leads III and aVF in EKG; chest pain; palpitations; difficulty breathing; rapid breathing; nausea; fatigue; sleep problem, sleep disorder, or sleep disturbance; constipation; and/or cognitive impairment.

K. Treatment and/or Prevention of Cognitive Impairment

The disclosure encompasses a method of treating, preventing, and/or slowing the onset or progression of cognitive impairment (CI) and/or a related symptom in a subject in need, the method comprising low dose nasal administration to the subject a therapeutically effective amount of at least one aminosterol, or a salt or derivative thereof. In one aspect, the CI is correlated with abnormal α-synuclein (αS) pathology and/or dopaminergic dysfunction.

Examples of conditions or disorders correlated with cognitive impairment, and which are also correlated with abnormal αS pathology, and/or dopaminergic dysfunction, include but are not limited to: (1) neurodegenerative diseases associated with neural cell death, (2) psychological or behavior disorders, and (3) cerebral and general ischemic disorders, as described in more detail below.

In one aspect, progression or onset of CI is slowed, halted, or reversed over a defined period of time following administration of the aminosterol or a salt or derivative thereof, as measured by a medically-recognized technique; and/or the CI is positively impacted by the aminosterol or a salt or derivative thereof, as measured by a medically-recognized technique. In another aspect, the positive impact on and/or progression of CI can be measured quantitatively or qualitatively by one or more techniques selected from the group consisting of computed tomography (CT), magnetic resonance imaging (MRI), magnetic resonance spectroscopy, functional MRI (fMRI), diffusion tensor imaging, single photon emission computed tomography (SPECT), and positron emission tomography (PET); and/or the progression or onset of CI can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In another aspect, the method results in improvement or resolution of CI or a CI-related symptom. The improvement or resolution can be measured using a clinically recognized scale or tool. The clinical scale or tool can be selected from the group consisting of Uniformed Parkinson's Disease Scale (UPDRS), Mini Mental State Examination (MMSE), Mini Mental Parkinson (MMP), Informant Questionnaire on Cognitive Decline in the Elderly (IQCODE), The 7-Minute Screen, Abbreviated Mental Test Score (AMTS), Cambridge Cognitive Examination (CAMCOG), Clock Drawing Test (CDT), General Practitioner Assessment of Cognition (GPCOG), Mini-Cog, Memory Impairment Screen (MIS), Montreal Cognitive Assessment (MoCA), Rowland Universal Dementia Assessment (RUDA), Self-Administered Gerocognitive Examination (SAGE), Short and Sweet Screening Instrument (SAS-SI), Short Blessed Test (SBT), St. Louis Mental Status (SLUMS), Short Portable Mental Status Questionnaire (SPMSQ), Short Test of Mental Status (STMS), Time and Change Test (T&C), Test Your Memory (TYM) test, and Addenbrooke's Cognitive Examination-Revised (ACER); and optionally the improvement in the CI or CI-related symptom can be at least about 3%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale or tool.

In another aspect, the CI correlated with abnormal αS pathology and/or dopaminergic dysfunction is related to or correlated with a neurodegenerative disease or neurological disease associated with neural cell death. For example, the neurodegenerative disease or neurological disease or related symptom associated with neural cell death can be: (a) selected from the group consisting of septic shock, intracerebral bleeding, subarachnoidal hemorrhage, multiinfarct dementia, inflammatory diseases, neurotrauma, peripheral neuropathies, polyneuropathies, metabolic encephalopathies, and infections of the central nervous system; or (b) selected from the group consisting of synucleopathies, Alzheimer's disease, Parkinson's disease, dementia with Lewy bodies, multiple system atrophy, Huntington's disease, multiple sclerosis, parkinsonism, amyotrophic lateral sclerosis (ALS), schizophrenia, Friedreich's ataxia, vascular dementia, spinal muscular atrophy, frontotemporal dementia, supranuclear palsy, progressive supranuclear palsy, progressive nuclear palsy, degenerative processes associated with aging, dementia of aging, Guadeloupian parkinsonism, spinocerebellar ataxia, hallucinations, stroke, traumatic brain injury, down syndrome, Gaucher's disease, Krabbe's disease (KD), lysosomal conditions affecting glycosphingolipid metabolism, cerebral palsy, and epilepsy.

In another aspect, the CI correlated with abnormal αS pathology and/or dopaminergic dysfunction is related to or correlated with a psychological or behavioral disorder. For example, the psychological or behavioral disorder can be selected from the group consisting of aberrant motor and obsessive-compulsive behaviors, sleep disorders, REM sleep behavior disorder (RBD), depression, major depressive disorder, agitation, anxiety, delirium, irritability, ADHD, apathy, bipolar disorder, disinhibition, addiction, illusion and delusions, amnesia, autism,

In another aspect, the CI correlated with abnormal αS pathology and/or dopaminergic dysfunction is related to or correlated with a cerebral ischemic disorder or a general ischemic disorder. For example, (a) the cerebral ischemic disorder can be selected from the group consisting of cerebral microangiopathy, intrapartal cerebral ischemia, cerebral ischemia during/after cardiac arrest or resuscitation, cerebral ischemia due to intraoperative problems, cerebral ischemia during carotid surgery, chronic cerebral ischemia due to stenosis of blood-supplying arteries to the brain, sinus thrombosis or thrombosis of cerebral veins, cerebral vessel malformations, and diabetic retinopathy; or (b) the general ischemic disorder can be selected from the group consisting of high blood pressure, high cholesterol, myocardial infarction, cardiac insufficiency, cardiac failure, congestive heart failure, myocarditis, pericarditis, perimyocarditis, coronary heart disease, angina pectoris, congenital heart disease, shock, ischemia of extremities, stenosis of renal arteries, diabetic retinopathy, thrombosis associated with malaria, artificial heart valves, anemias, hypersplenic syndrome, emphysema, lung fibrosis, and pulmonary edema.

The CI-related symptom can be, for example, cognitive impairment as determined by an IQ score; cognitive impairment as determined by a memory or cognitive function test; decline in thinking and reasoning skills; confusion; poor motor coordination; loss of short term memory; loss of long term memory; identity confusion; impaired judgement; forgetfulness; depression; anxiety; irritability; obsessive-compulsive behavior; apathy and/or lack of motivation; emotional imbalance; problem solving ability; impaired language; impaired reasoning; impaired decision-making ability; impaired ability to concentrate; impaired communication; impaired ability to conduct routine tasks such as cooking; self-care, including feeding and dressing; constipation; neurodegeneration; sleep problem, sleep disorder, and/or sleep disturbance; hypertension; hypotension; sexual dysfunction; cardiovascular disease; cardiovascular dysfunction; difficulty with working memory; gastrointestinal (GI) disorders; attention deficit and hyperactivity disorder; seizures; urinary dysfunction; difficulty with mastication; vision problems; and/or muscle weakness.

L. Treatment and/or Prevention of Constipation

In one aspect, encompassed is a method of treating constipation and/or a constipation-related symptom in a subject in need, comprising low dose nasal administration an aminosterol or a salt or derivative thereof to the subject.

The constipation-related symptom can be, for example, frequency of constipation; duration of constipation symptoms; frequency of bowel movements; fecal incontinence/encopresis; abdominal pain; abdominal distension or bloating; abdominal discomfort; stomach cramps; stool consistency; painful defecation/rectal pain with bowel movement; rectal burning during or after bowel movement; rectal bleeding or tearing during or after a bowel movement; ease of defecation/passing stool; straining during defecation and/or straining or squeezing to try to pass bowel movements; incomplete evacuation or bowel movement; unsuccessful attempts at evacuation; sensation of incomplete bowel evacuation; sensation of anorectal obstruction/blockage; bowel movements that were too hard; bowel movements that were too small; change in amount of gas passed rectally; less frequent bowel movements; oozing liquid stool; rectal fullness or pressure; small stool size; urge but inability to pass stool; and/or personal judgement of constipation.

In one aspect, the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%. The improvement can be measured using a clinically recognized scale or tool.

In another aspect, the constipation-related symptom is frequency of bowel movements, and the improvement or resolution comprises a desired rate of complete spontaneous bowel movement (CSBM) or spontaneous bowel movement (SBM). In another aspect, the constipation-related symptom is frequency of bowel movements, and the improvement or resolution comprises a rate of CSBM or SBM in the subject of one or more CSBM or SBM per week, 2 or more CSBM or SMB per week, or 3 or more CSBM or SBM per week. In another aspect, the improvement or resolution comprises an increase in bowel activity, an induction of nausea, an induction of secretory diarrhea, or any combination thereof.

In one aspect, the subject is suffering from a disorder of gastrointestinal motility, and in another aspect he subject is suffering from a condition or disorder such as chronic idiopathic constipation, Irritable bowel syndrome, Opioid-induced constipation, or Inflammatory Bowel Disease.

In another aspect, the subject is suffering from a neurodegenerative disease. For example, the neurodegenerative disease can be Parkinson's Disease, Alzheimer's disease (AD), Huntington's chorea and/or Huntington's disease, Multiple Sclerosis, Amyotorphic Lateral Sclerosis (ALS), multiple system atrophy (MSA), schizophrenia, Friedreich's ataxia, vascular dementia, Lewy Body dementia or disease, spinal muscular atrophy, supranuclear palsy, frontotemporal dementia, progressive nuclear palsy, Guadeloupian Parkinsonism, spinocerebellar ataxia, autism, dementia of aging, neuropathy of diabetes, peripheral sensory neuropathy, cerebral palsy, epilepsy, diabetic neuropathy, traumatic head and/or spine injury, stroke, or depression.

M. Treatment and/or Prevention of Erectile Dysfunction

In one aspect, encompassed is a method of treating, preventing, and/or slowing the onset or progression of erectile dysfunction (ED) and/or a related symptom in a subject in need comprising low dose intranasal administration to the subject a therapeutically effective amount of at least one aminosterol, or a salt or derivative thereof.

In one aspect, the method results in a decrease in the number of instances in which the subject cannot attain erection, and the decrease in number of instances in which the subject cannot attain erection comprises a reduction in number of instances in which the subject cannot attain erection over a defined period of time. In another aspect, the method results in a decreased severity of ED over a defined period of time, wherein the decreased severity of ED is measured by a medically recognized technique selected from the group consisting of bone-pressed erect length (BPEL) measurement, girth measurement, Erection Hardness Scale (EHS), and International Index of Erectile Function (IIEF).

In another aspect, (a) progression or onset of ED is slowed, halted, or reversed over a defined period of time following administration of the fixed escalated dose of the aminosterol or a salt or derivative thereof, as measured by a medically-recognized technique; and/or (b) the ED is positively impacted by the fixed escalated dose of the aminosterol or a salt or derivative thereof, as measured by a medically-recognized technique. Each defined period of time can independently be selected from the group consisting of about 1 day to about 10 days, about 10 days to about 30 days, about 30 days to about 3 months, about 3 months to about 6 months, about 6 months to about 12 months, and about greater than 12 months. The positive impact and/or progression of ED can be measured quantitatively or qualitatively by one or more techniques selected from the group consisting of bone-pressed erect length (BPEL) measurement, girth measurement, Erection Hardness Scale (EHS), and International Index of Erectile Function (IIEF); and/or the progression or onset of ED can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by the one or more techniques.

In yet another aspect, the aminosterol or a salt or derivative thereof reverses dysfunction caused by the ED and treats, prevents, improves, and/or resolves the ED-related symptom. The improvement or resolution of the ED symptom can measured using a clinically recognized scale or tool; and/or the improvement in the ED symptom can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale.

The ED symptom can be, for example, (a) a symptom from the International Index of Erectile Function (IIEF) selected from the group consisting of likelihood of getting an erection during sexual activity, likelihood that erections are hard enough for penetration, ability to maintain erection after penetration, ability to maintain erection to completion of intercourse, satisfaction with intercourse attempts, likelihood of ejaculation during sexual intercourse or stimulation, likelihood of orgasm during sexual intercourse or stimulation, prevalence of sexual desires, intensity of sexual desires, satisfaction with sexual relationship with partner, and confidence level in ability to get and maintain erection; (b) constipation; (c) sleep disorder or sleep disturbance; (d) neurodegeneration; (e) cognitive impairment; (f) bone-pressed erect length (BPEL) measurement; (g) hardness as specified in the Erection Hardness Scale (EHS); (h) erect penile girth; (i) high blood pressure; (j) diabetes; (k) atherosclerosis; (1) heart disease; (m) high cholesterol; (n) multiple sclerosis; (o) obesity; (p) depression; and (q) anxiety.

### IV. Definitions

The following definitions are provided to facilitate understanding of certain terms used throughout this specification.

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

As used in the description of the invention and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein the term “Aminosterol 1436” encompasses Aminosterol 1436 or a derivative or salt thereof, an isomer or prodrug of Aminosterol 1436.

As used herein, the phrase “therapeutically effective amount” means a dose of Aminosterol 1436, or a salt or derivative thereof, that provides the specific pharmacological effect for which the compound or compounds are being administered. It is emphasized that a therapeutically effective amount will not always be effective in achieving the intended effect in a given subject, even though such dose is deemed to be a therapeutically effective amount by those of skill in the art. For convenience only, exemplary dosages are provided herein. Those skilled in the art can adjust such amounts in accordance with standard practices as needed to treat a specific subject. The therapeutically effective amount may vary based on the route of administration and dosage form, the age and weight of the subject, and/or the severity of the subject's condition. For example one of skill in the art would understand that the therapeutically effective amount for treating a small individual may be different from the therapeutically effective amount for treating a large individual.

The term “administering” as used herein includes prescribing for administration as well as actually administering, and includes physically administering by the subject being treated or by another.

As used herein “subject” or “patient” or “individual” refers to any subject, patient, or individual, and the terms are used interchangeably herein. In this regard, the terms “subject,” “patient,” and “individual” includes mammals, and, in particular humans.

### V. Examples

The following examples are provided to illustrate the present invention. It should be understood, however, that the invention is not to be limited to the specific conditions or details described in these examples. Throughout the specification, any and all references to a publicly available document, including a U.S. patent, are specifically incorporated by reference.

### Example 1

The purpose of this example was to evaluate the in vivo distribution of Aminosterol 1436 following intracerebroventricular (ICV) and intravenous (IV) administration to rats. ICV injection is an invasive injection technique of substances directly into the cerebrospinal fluid in cerebral ventricles to bypass the blood brain barrier. The results described below detail how aminosterols such as Aminosterol 1436 localize in the brain following in vivo administration, regardless of the route of administration.

Radiolabeled Aminosterol 1436 was injected into rats by two different forms of administration: ICV and IV administration. Surprisingly, it was found that following both forms of administration, Aminosterol 1436 localized to the same portion of the brain.

Intravenously administered Aminosterol 1436 localized in the hypothalamus. See FIG. 1B. In particular, FIG. 1B shows two panels of the distribution of ³H-Aminosterol 1436 in rat forebrain following IV administration to rats. The specific areas of ³H-Aminosterol 1436 localization include the regions below the third ventricle, in the mesiobasal hypothalamus, periventricular (PVN) and arcuate nuclei (FIG. 1C); these parts of the brain control feeding behavior and appetite and have significant involvement with neurogenesis.

Intracerebroventricularly (ICV) administered Aminosterol 1436 (ICV) localized to the same regions of the brain. See FIG. 1A. From the ventricular cerebrospinal fluid, Aminosterol 1436 is absorbed through the choroid plexus of the ventricles and vascularly transported to the same regions. In particular, FIG. 1A shows two panels of the distribution of ³H-Aminosterol 1436 binding in rat forebrain following ICV administration. The drug distribution parallels that seen with IV administration.

This example clearly demonstrates that the route of administration does not impact the site of in vivo localization of an aminosterol such as Aminosterol 1436.

### Example 2

The purpose of this example was to evaluate the in vivo distribution of the aminosterol Aminosterol 1436 following intraperitoneal administration (IP) and ICV administration, and to determine the impact the drug has on food intake and body weight when administered IP and ICV.

Additional data regarding in vivo distribution and the effect of the route of administration for an aminosterol is shown in FIG. 2. In particular, FIG. 2A shows the in vivo distribution of the aminosterol Aminosterol 1436 administered IP or ICV as compared to vehicle (administered IP) in the Arc (arcuate nucleus of the hypothalamus), PVN (paraventricular nucleus of the hypothalamus), LH (lateral hypothalamus), VMN (ventromedial nucleus of the hypothalamus), CcA (central amygdala), and NTS (Nucleus Tractus Solitarius, a longitudinal structure in the medulla). The data in FIG. 2A clearly show similar in vivo distribution for all areas of the brain evaluated for Aminosterol 1436 administered IP or ICV.

FIG. 2B shows the effect on food intake over a 10 day period for animals administered vehicle ICV, vehicle IP, Aminosterol 1436 at 10 and 40 μg ICV, and Aminosterol 1436 at 5 mg/kg intraperitoneal injection (IP). This experiment is relevant as the areas of the brain where the aminosterol Aminosterol 1436 localized upon administration are known to control feeding behavior and appetite. The results shown in FIG. 2B demonstrate that an aminosterol such as Aminosterol 1436 administered either IP or ICV has a significant impact on food intake, which is consistent with the area of localization of the drug in the brain.

Finally, FIG. 2C shows the percent change in body weight for the experiment detailed in FIG. 2B, with a decrease in body weight correlating with a decrease in food intake shown in FIG. 2B.

This example clearly demonstrates that the route of administration does not impact the site of in vivo localization of an aminosterol such as Aminosterol 1436.

### Example 3

The purpose of this example was to evaluate the in vivo distribution and function of aminosterols, such as Aminosterol 1436 and squalamine, following intranasal administration. This experiment relates to the amount of drug needed to obtain a therapeutic result, based on an IN route of administration. The results described below detail that aminosterols such as Aminosterol 1436 act at the level of the hypothalamus following in vivo administration, regardless of the route of administration.

Prior to the present invention, it was assumed that intranasal administration of an aminosterol such as Aminosterol 1436 would result in first systemic drug circulation, followed by dilution in various organs around the body, and that some small fraction of the aminosterol would eventually reach the hypothalamus. Alternatively, it was also believed that an intranasally administered aminosterol would be transported across the olfactory epithelium, enter the perineural space and track along the olfactory nerves and find its way into the cerebrospinal fluid (CSF), and subsequently to the hypothalamus.

Instead, it was surprisingly found that an intranasally administered aminosterol, such as Aminosterol 1436, was not absorbed into the CSF, but rather was readily absorbed into the blood. In fact, it was unexpectedly found that intranasal administration of an aminosterol such as Aminosterol 1436 produced 10 times higher blood levels of Aminosterol 1436 than peripherally injected Aminosterol 1436. See e.g., FIG. 8. In addition, administration of the aminosterol squalamine was also found to result in a similar distribution pattern.

Specifically, FIG. 3A shows the plasma concentration (ng/mL) vs time for squalamine lactate after 0.5 mg/kg administered intranasally (IN) in Sprague Dawley® (SD) rats, and FIG. 3C shows the CSF concentration (ng/mL) vs time profile for squalamine lactate following 0.5 mg/kg administered IN to SD rats. Similarly, FIG. 3B shows the plasma concentration (ng/mL) vs time for Aminosterol-1436 (“MSI-1436”) after 0.5 mg/kg administered IN in SD rats, and FIG. 3D shows the CSF concentration (ng/mL) vs time profile for Aminosterol 1436 following 0.5 mg/kg administered IN to SD rats. No squalamine lactate or Aminosterol 1436 was found in CSF following intranasal administration.

Pharmacokinetic information for these tests can be found in the tables below.

TABLE 2
Summary of Aminosterol 1436 IN pharmacokinetic
parameters in plasma following 0.5 mg/kg IN dose

| PK parameters | Unit | Mean |
|---|---|---|
| T_{1/2} | h | 7.59 |
| Tₘₐₓ | h | 4.00 |
| Cₘₐₓ | ng/mL | 1677 |
| AUCₗₐₛₜ | h * ng/mL | 9371 |
| AUC_{Inf} | h * ng/mL | 17898 |
| AUC__{%Extrap}_obs | % | 47.6 |
| MRT_{Inf}_obs | h | 11.1 |
| AUCₗₐₛₜ/D | h * mg/mL | 18742 |
| F | % | NA |

Data for Table 2 is also depicted in FIG. 3B.

TABLE 3
Summary of Aminosterol 1436 pharmacokinetic
parameters in CSF following 0.5 mg/kg IN dose

| PK parameters | Unit | Mean |
|---|---|---|
| T_{1/2} | h | NA |
| Tₘₐₓ | h | NA |
| Cₘₐₓ | ng/mL | NA |
| AUCₗₐₛₜ | h * ng/mL | NA |
| AUC_{Inf} | h * ng/mL | NA |
| AUC__{%Extrap}_obs | % | NA |
| MRT_{Inf}_obs | h | NA |
| AUCₗₐₛₜ/D | h * mg/mL | NA |
| F | % | NA |

Data for Table 3 is also depicted in FIG. 3D.

TABLE 4
Summary of Squalamine Lactate pharmacokinetic
parameters in plasma following 0.5 mg/kg IN dose

| PK parameters | Unit | Mean |
|---|---|---|
| T_{1/2} | H | 4.55 |
| Tₘₐₓ | H | 1.00 |
| Cₘₐₓ | ng/mL | 1001 |
| AUCₗₐₛₜ | h * ng/mL | 5296 |
| AUC_{Inf} | h * ng/mL | 7863 |
| AUC__{%Extrap}_obs | % | 32.7 |
| MRT_{Inf}_obs | H | 7.12 |
| AUCₗₐₛₜ/D | h * mg/mL | 10592 |
| F | % | NA |

Data for Table 4 is also depicted in FIG. 3A.

TABLE 5
Summary of Squalamine Lactate pharmacokinetic
parameters in CSF following 0.5 mg/kg IN dose

| PK parameters | Unit | Mean |
|---|---|---|
| T_{1/2} | h | NA |
| Tₘₐₓ | h | NA |
| Cₘₐₓ | ng/mL | NA |
| AUCₗₐₛₜ | h * ng/mL | NA |
| AUC_{Inf} | h * ng/mL | NA |
| AUC__{%Extrap}_obs | % | NA |
| MRT_{Inf}_obs | h | NA |
| AUCₗₐₛₜ/D | h * mg/mL | NA |
| F | % | NA |

Data for Table 5 is also depicted in FIG. 3C.

Based on the data detailed in FIGS. 3A-D, and Tables 3-6, it was concluded that the administration of aminosterols, such as Aminosterol 1436 and squalamine, results in the drug crossing the nasal epithelium and being absorbed into a very rich submucosal capillary network, from which the drug then drained into the cavernous sinus. Within the cavernous sinus, arterial and venous blood are admixed. Blood from the cavernous sinus is pumped by the internal carotid artery passing through it into the microvasculature of the brain, specifically the microvasculature of the hypothalamus, and more specifically the mesiobasal hypothalamus.

It so happens that the vascular network in the nasal cavity, the cavernous sinuses right behind the nasal cavity and the mesiobasal hypothalamus, are all incredibly close to each other (e.g., no more than 1-2 cm apart). See FIG. 4, which shows the structure of the hypothalamus, including (1) the hypophysis and (2) intercavernous sinus, (3) the internal carotid artery and internal carotid vein, (4) specific nerves, including the oculomotor nerve, trochlear nerve, ophthalmic nerve, abducens nerve, and maxillary nerve, (5) ganglions, including the sphenopalatine ganglion and upper cervical ganglion, and (6) the cavernous and carotid sympathetic plexus. FIG. 4 clearly shows that the hypothalamus is located very close to the cavernous sinus. A close up of this structure is shown in FIG. 5.

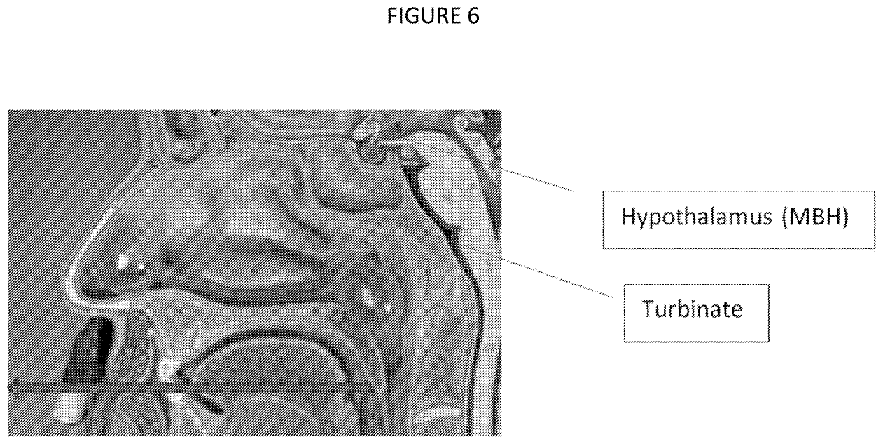

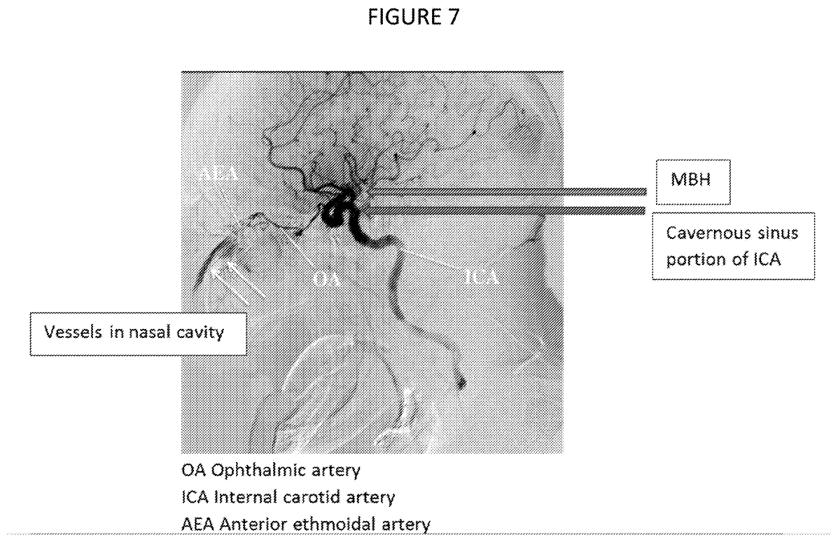

FIG. 6 shows a side-on picture through the nasal cavity showing the turbinates which are highly vascularized. This Fig. also shows how close the mesial basal hypothalamus is to this large cavity. Similarly, FIG. 7 shows the vessels in the nasal cavity, with the cavernous sinus portion of the internal carotid artery (ICA) and the medial basal hypothalamus (MBH), ophthalmic artery (OA), internal carotid artery (ICA), and anterior ethmoidal artery (AEA) identified on the figure.

This structure of the brain provides for incredibly rapid transport of an aminosterol directly into the site at which it acts, e.g., the hypothalamus. Thus, minute amounts of an aminosterol compound administered intranasally are sufficient to produce a pharmacologic effect because they are directly delivered into a tiny compartment very close to the hypothalamus. This was not known prior to the present invention.

### Example 4

The purpose of this example was to evaluate and compare the impact on weight following administration of an aminosterol such as Aminosterol 1436 to mice via IP or IN.

Mice were administered: (i) intraperitoneally 1 mg/kg or 10 mg/kg of Aminosterol 1436, (ii) intranasally 0.4 mg/kg of Aminosterol 1436, or (iii) a saline control administered IN. See FIG. 8. Weight of the mice was then measured for 10 days post-administration.

The results shown in FIG. 8 demonstrate that 1 mg/kg of intraperitoneally administered Aminosterol 1436 compared similarly to the intranasally administered saline control. However, surprisingly, the IN administered Aminosterol 1436 in the amount of 0.4 mg/kg resulted in a decrease in the weight of the mice in an amount comparable to 10 mg/kg of intraperitoneally administered Aminosterol 1436. See FIG. 8.

Thus, surprisingly, intranasal administration of an aminosterol in an animal model was found to be at least 10 fold more potent than intraperitoneal administration of the same aminosterol.

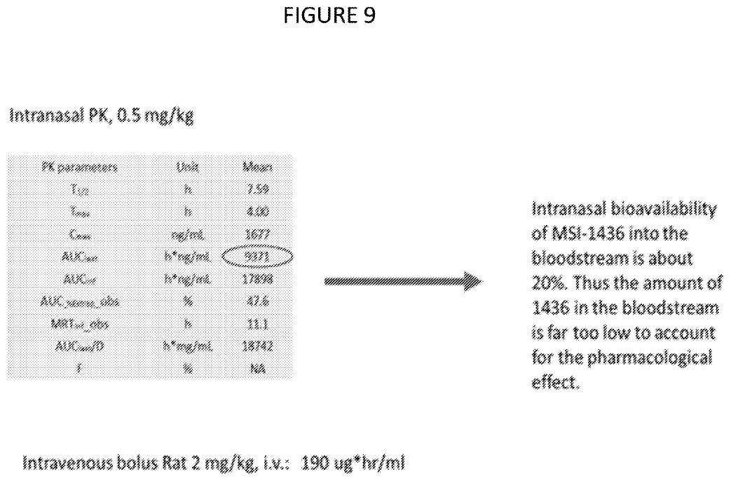

However, as pharmacokinetic testing indicates, the intranasal bioavailability of the aminosterol Aminosterol 1436 in the bloodstream is about 20%. In particular, FIG. 9 shows the PK profile in a rat following IV bolus injection of 2 mg/kg, 190 μg hr/ml of Aminosterol 1436, as compared to IN administration of 0.5 mg/kg Aminosterol 1436. The pK profile shows that IN bioavailability of Aminosterol 1436 is about 20%. Thus, the amount of aminosterol in the bloodstream is too low to account for the observed pharmacological effect. This means that when administered IN, an aminosterol is having a pharmacological effect via a mechanism other than via plasma concentration of the drug.

### Example 5

The purpose of this example was to establish the tolerability of IN effective doses and to identify potential formulations for use in humans.

Initially, 20 mg of lactose powder was intranasally administered to a human subject. No pharmacological effect was observed from the IN administered lactose powder. Next, 4 mg of Aminosterol 1436 hydrochloride powder in 20 mg lactose powder was IN administered. The IN administration of the Aminosterol 1436 plus lactose powder elicited some stinging and lacrimation which lasted about 30 minutes.

Next, liquid formulations of the phosphate salt of Aminosterol 1436 were tested. A phosphate salt of Aminosterol 1436 was chosen because the material is quite insoluble, and was expected to release slowly and minimize stinging sensation upon IN administration. 0.05, 0.1 and 1 mg/ml liquid solutions of Aminosterol 1436 phosphate with the 1436 salt suspended in normal saline (saline nasal spray) were tested. Minimal discomfort was observed upon administration of these liquid formulations and a delayed tingling sensation was observed only at the highest concentration (1 mg/ml) and 1-2 minute post-administration. In other words, the formulations were observed to be very tolerable for human administration.

2 mg/ml of Aminosterol 1436 phosphate was then suspended in a 2:1 mixture of normal saline: glycerine. In this manner, the phosphate salt was better suspended in solution to prevent it from falling to the bottom of the liquid. Following intranasal administration of 0.2 cc, this 2 mg/ml formulation comprising glycerine was observed to also be quite tolerable and barely any stinging was observed 1-2 minutes post-administration.

Thus, doses which are likely to be efficacious in the human brain were found to be tolerable when IN administered.

### Example 6

This prophetic example describes an exemplary method of delaying and/or preventing progression and/or onset of age-related neurodegeneration in a subject, comprising intranasally administering a low dose of a pharmaceutical composition comprising a therapeutically effective amount of an aminosterol or a pharmaceutically acceptable salt or derivative thereof to a subject in need.

One or more subjects are intranasally given a low dose of a pharmaceutical composition comprising an aminosterol, such as Aminosterol 1436, squalamine, or a combination thereof. In an exemplary method, the composition is administered daily intransally at a low dosage of about 0.1 to about 20 mg.

Age-related neurodegeneration is evaluated prior to treatment to form a baseline, using a medically recognized technique, and then periodically following initiation of treatment. At least one control subject, of the same sex and age as the tested subjects, does not receive an aminosterol treatment.

The treated subjects are expected to show slowed progression and/or onset of age-related neurodegeneration as compared to the untreated control subject. It is expected that administration of an aminosterol, such as Aminosterol 1436 and/or squalamine, will result in slowing progression and/or onset of age-related neurodegeneration by about 5% or more. This method is applicable, for example, to treating and/or preventing neurodegeneration associated with normal aging as well as neurodegeneration associated with disease, such as Alzheimer's disease.

### Example 7

This prophetic example describes an exemplary method of treating or preventing a sleep disorder or sleep disturbance in a subject, comprising intranasally administering a low dose of a pharmaceutical composition comprising a therapeutically effective amount of an aminosterol or a pharmaceutically acceptable salt or derivative thereof to a subject in need.

One or more human subjects are intranasally given a low dose of a pharmaceutical composition comprising an aminosterol, such as Aminosterol 1436, squalamine, or a combination thereof. In an exemplary method, the composition is administered daily intransally at a low dosage of about 0.1 to about 20 mg.

The methods of the invention are expected to positively impact sleep disorders, including but not limited to insomnia, narcolepsy, restless leg syndrome, parasomnias, circadian rhythm sleep disorders, and non-24 hour sleep-wake disorder.

### Example 8

This prophetic example describes an exemplary method of treating autism. The method comprising intranasally administering a low dose of a pharmaceutical composition comprising a therapeutically effective amount of an aminosterol or a pharmaceutically acceptable salt or derivative thereof to a subject in need.

One or more human subjects can be intranasally given a suitable low dosage of an aminosterol, such as Aminosterol 1436, squalamine, or a combination thereof. An exemplary low dosage can be, for example, about 0.1 to about 20 mg administered intranasally daily.

Characteristics of autism are evaluated prior to treatment to form a baseline, using a medically recognized technique, and then periodically following initiation of treatment. At least one control subject, of the same sex and age as the tested subjects, does not receive aminosterol treatment.

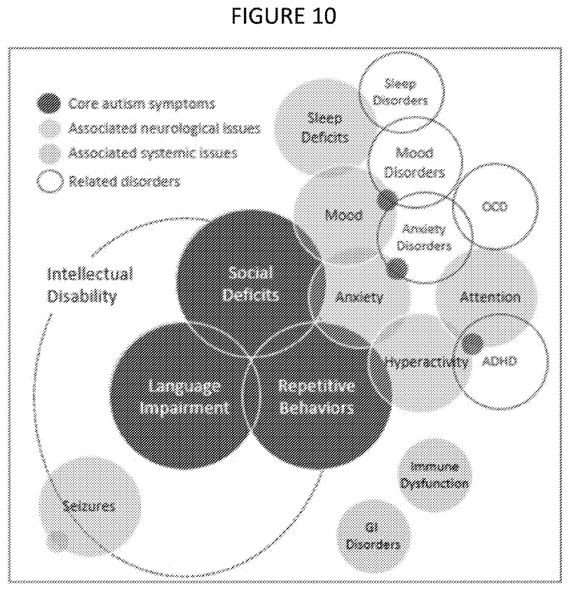

Examples of characteristics of autism that can be positively affected by the method of the invention include, for example, motor skills, sleep deficits, communication and language skills, social interaction, attention, anxiety, OCD behaviors, and mood. See e.g., FIG. 10, which lists characteristics of autism. Potentially all of these characteristics can be positively impacted by the methods of the invention.

### Example 9

This prophetic example describes an exemplary method of treating depression. The method comprising intranasally administering a low dose of a pharmaceutical composition comprising a therapeutically effective amount of an aminosterol or a pharmaceutically acceptable salt or derivative thereof to a subject in need.

One or more human subjects can be intranasally given a suitable low dosage of an aminosterol, such as Aminosterol 1436, squalamine, or a combination thereof. An exemplary low dosage can be, for example, about 0.1 to about 20 mg administered intranasally daily.

Characteristics of depression are evaluated prior to treatment to form a baseline, using a medically recognized technique, and then periodically following initiation of treatment. At least one control subject, of the same sex and age as the tested subjects, does not receive aminosterol treatment.

Examples of characteristics of depression that can be positively affected by the method of the invention include, but are not limited to, trouble concentrating, remembering details, and making decisions; fatigue; feelings of guilt, worthlessness, and helplessness; pessimism and hopelessness; insomnia, early-morning wakefulness, or sleeping too much; irritability; restlessness; loss of interest in things once pleasurable, including sex; overeating, or appetite loss; aches, pains, headaches, or cramps that won't go away; digestive problems that don't get better, even with treatment; persistent sad, anxious, or “empty” feelings; suicidal thoughts or attempts. Potentially all of these characteristics can be positively impacted by the methods of the invention.

### Example 10

This prophetic example describes an exemplary method of treating constipation. The method comprising intranasally administering a low dose of a pharmaceutical composition comprising a therapeutically effective amount of an aminosterol or a pharmaceutically acceptable salt or derivative thereof to a subject in need.

One or more human subjects can be intranasally given a suitable low dosage of an aminosterol, such as Aminosterol 1436, squalamine, or a combination thereof. An exemplary low dosage can be, for example, about 0.1 to about 20 mg administered intranasally daily.

Characteristics of constipation are evaluated prior to treatment to form a baseline, using a medically recognized technique, and then periodically following initiation of treatment. At least one control subject, of the same sex and age as the tested subjects, does not receive aminosterol treatment.

Examples of characteristics of constipation that can be positively affected by the method of the invention include, but are not limited to, frequency of constipation, duration of constipation symptoms, bowel movement frequency, stool consistency, abdominal pain, abdominal bloating, incomplete evacuation, unsuccessful attempts at evacuation, pain with evacuation, and straining with evacuation. Potentially all of these characteristics can be positively impacted by the methods of the invention.

### Example 11

This prophetic example describes an exemplary method of improving cognitive ability, which has been negatively impacted by age or disease. The method comprising intranasally administering a low dose of a pharmaceutical composition comprising a therapeutically effective amount of an aminosterol or a pharmaceutically acceptable salt or derivative thereof to the subject.

One or more human subjects can be intranasally given a low dosage of an aminosterol, such as Aminosterol 1436, squalamine, or a combination thereof. An exemplary low dosage can be, for example, about 0.1 to about 20 mg administered intranasally daily.

Cognitive ability can be evaluated for each subject prior to initial aminosterol dosing to establish a baseline using a conventional cognitive ability test. Following initiation of aminosterol dosing, the cognitive ability test is repeated periodically to measure improvement. It is anticipated that cognitive ability will improve following aminosterol dosing by about 5% or more.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

### REFERENCES

Ahima et al., “Appetite suppression and weight reduction by a centrally active aminosterol.” Diabetes, 51(7): 2099-104 (2002).
Akhter et al., “Squalamine, a novel cationic steroid, specifically inhibits the brush-border Na+/H+ exchanger isoform NHE3.” Am. J. Physiol., 276(1 Pt 1): C136-44 (1999).
Alexander et al., “Membrane surface charge dictates the structure and function of the epithelial na+/h+ exchanger. EMBO J., 30:679-691 (2011)
Bhargava et al., “A phase I and pharmacokinetic study of squalamine, a novel antiangiogenic agent, in patients with advanced cancers,” Clin. Cancer Res., 7(12): 3912-9 (2001).
Blagosklonny, M. V., “Big mice die young but large animals live longer,” Aging (Albany N.Y.) 5, 227-233 (2013).
Connolly et al., “Squalamine lactate for exudative age-related macular degeneration,” Ophthalmol. Clin. North Am., 19:381-91 (2006).
Delgado et al., “Neuroprotective effect of vasoactive intestinal peptide (VIP) in a mouse model of Parkinson's disease by blocking microglial activation.” Faseb. J., 17(8): 944-6 (2003).
Genaidy et al., “Effect of squalamine on iris neovascularization in monkeys.” Retina, 22(6): 772-8 (2002).
Genesis, A., “Squalamine trial for the treatment of fibrodysplasia ossificans progressiva initiated,” Angiogenesis Weekly, 8:45 (2002).
Gonzalez-Rey et al., “Therapeutic effect of vasoactive intestinal peptide on experimental autoimmune encephalomyelitis: down-regulation of inflammatory and autoimmune responses,” Am. J. Pathol., 168(4): 1179-88 (2006)
Gressens et al., “Vasoactive intestinal peptide prevents excitotoxic cell death in the murine developing brain,” J. Clin. Invest., 100(2): 390-7 (1997).
Hao et al., “A Phase I and pharmacokinetic study of squalamine, an aminosterol angiogenesis inhibitor,” Clin. Cancer Res., 9(7): 2465-71 (2003).
Herbst et al., “A phase I/IIA trial of continuous five-day infusion of squalamine lactate (MSI-1256F) plus carboplatin and paclitaxel in patients with advanced non-small cell lung cancer,” Clin. Cancer Res., 9(11): 4108-15 (2003).
T. Hicklin, “Brain cells that influence aging,” NIH Research Matters (Aug. 15, 2017), https://www.nibov/news-events/ni-research-natters/brain-cells-influence-aing (accessed on Feb. 14, 2018).
Higgins et al., “Squalamine improves retinal neovascularization,” Invest. Ophthalmol. Vis. Sci., 41(6): 1507-12 (2000).
Higgins et al., “Regression of retinopathy by squalamine in a mouse model,” Pediatr. Res., 56(1): 144-9 (2004).
Lantz et al., “Inhibition of PTP1B by trodusquemine (MSI-1436) causes fat-specific weight loss in diet-induced obese mice,” Obesity (Silver Spring), 18:1516-1523 (2010).
Li et al., “Squalamine and cisplatin block angiogenesis and growth of human ovarian cancer cells with or without HER-2 gene overexpression,” Oncogene, 21(18): 2805-14 (2002).
Li et al., “GLP-1 receptor stimulation preserves primary cortical and dopaminergic neurons in cellular and rodent models of stroke and Parkinsonism,” Proc. Natl. Acad Sci. USA, 106(4): 1285-90 (2009).
MacDonald, D. (1995). “Squalamine for STDs.” Abstract no F7 35th ICAAC conference.
Miller et al., “Big mice die young: early life body weight predicts longevity in genetically heterogeneous mice,” Aging Cell, 1: 22-29 (2002).
Moore et al., “Squalamine: an aminosterol antibiotic from the shark,” Proc. Natl. Acad Sci. USA, 90(4): 1354-8 (1993).
Rao et al., “Aminosterols from the dogfish shark Squalus acanthias,” J. Nat. Prod, 63(5): 631-5 (2000).
Salmi et al., “New stereoselective titanium reductive amination synthesis of 3-amino and polyaminosterol derivatives possessing antimicrobial activities,” Eur. J. Med Chem., 43(3): 540-7 (2008).
Salmi et al., “Squalamine: an appropriate strategy against the emergence of multidrug resistant gram-negative bacteria?” PLoS ONE, 3(7): e2765 (2008).
Schiller, J. H. and G. Bittner, “Potentiation of platinum antitumor effects in human lung tumor xenografts by the angiogenesis inhibitor squalamine: effects on tumor neovascularization,” Clin. Cancer Res., 5(12): 4287-94 (1999).
Selinsky et al., “Squalamine is not a proton ionophore,” Biochim. Biophys. Acta., 1464(1): 135-41 (2000).
Selinsky et al., “The aminosterol antibiotic squalamine permeabilizes large unilamellar phospholipid vesicles,” Biochim. Biophys. Acta., 1370(2): 218-34 (1998).
Sills et al., “Squalamine inhibits angiogenesis and solid tumor growth in vivo and perturbs embryonic vasculature,” Cancer Res., 58(13): 2784-92 (1998).
Sokoloff et al., “Adjunctive therapy for men with high risk localized and locally advanced prostate cancer: targeting disseminated tumor cells,” J. Urol., 172(6 Pt 2): 2539-44 (2004).
Steinberg, B. E. and S. Grinstein, “Pathogen destruction versus intracellular survival: the role of lipids as phagosomal fate determinants,” J. Clin. Invest., 118(6): 2002-11 (2008).
Sumioka et al., “TARP phosphorylation regulates synaptic AMPA receptors through lipid bilayers,” Neuron, 66(5): 755-67 (2009).
Tirassa et al., “CCK-8 prevents the development of kindling and regulates the GABA and NPY expression in the hippocampus of pentylenetetrazole (PTZ)-treated adult rats,” Neuropharmacology, 48(5): 732-42 (2005).
US 2005/0261508A1 for “Aminosterol Compounds useful as inhibitors of the sodium/proton exchanger (NHE), pharmaceutical methods, and compositions employing such inhibitors, and processes for evaluating the NHE-inhibitory efficacy of compounds,” Zasloff et al., Published 11/24/05.
US 2006/0166950A1 for “Treatment of neovascularization disorders with squalamine,” Zasloff et al., Published Jun. 27, 2006
US 2006/0183928A1 for “Aminosterol Compounds useful as inhibitors of the sodium/proton exchanger (NHE), pharmaceutical methods, and compositions employing such inhibitors, and processes for evaluating the NHE-inhibitory efficacy of compounds”, Published Aug. 17, 2006
US 2007/10504A1 for “Polymorphic and Amorphous salt forms of squalamine dilactate” Chellquist, Doubleday, Gilbert, Zhang, McLane, Armbruster, Levitt, Published Jan. 11, 2007
US 2011/0097303 for “Methods and Compositions for Treating and Preventing Viral Infections,” published Apr. 28, 2011, Zasloff); U.S. (2011) Ser. No. 12/913,648
U.S. Pat. No. 5,192,756 for “Aminosterol antibiotic,” Zasloff, Moore, Wehrli, Issued Mar. 9, 1993
U.S. Pat. No. 5,637,691 (1993) for “Steroid derivatives, pharmaceutical compositions containing them, and their use as antibiotics and disinfectants”, Frye, Zasloff, Kinney, Moriarty.
U.S. Pat. No. 5,721,226 (1998) for “Methods for treating angiogenesis using squalamine and squalamine steroid derivatives,” Frye, Zasloff, Kinney, Moriarty, Collins
U.S. Pat. No. 5,733,899 (1998) for “Methods for treating infections using steroid based pharmaceutical compositions,” Frye, Zasloff, Kinney, Moriarty, Collins
U.S. Pat. No. 5,763,430 (1998) for “Method of treating a viral infection by administering a steroid compound,” Zasloff.
U.S. Pat. No. 5,792,635 (1998) for “Method of inhibiting the sodium-proton exchanger NHE3 and method of inhibiting growth by administering squalamine,” Zasloff.
U.S. Pat. No. 5,795,885 (1998) for “Method of Inhibiting proliferation of cells by administering an aminosterol compound,” Zasloff, Shinnar, Kinney, Anderson, Williams,
McLane.
U.S. Pat. No. 5,834,453 (1998) for “Methods for the manufacture and use of antimicrobial sterol conjugates,” Regen (Leheigh Univ).
U.S. Pat. No. 5,840,740 (1998) for “Aminosterol compounds and a method of treating infection using the aminosterol compounds,” Zasloff, Shinnar, Kinney, Rao.
U.S. Pat. No. 5,840,936 (1998) for “Aminosterol compounds useful as inhibitors of the sodium/proton exchanger(NHE),” Zasloff, Shinnar, Rao, Kinney.
U.S. Pat. No. 5,847,172 (1998) for “Certain Aminosterol compounds and Pharmaceutical compositions including these compounds,” Zasloff, Shinnar, Kinney, Jones.
U.S. Pat. No. 5,856,535 (1999) for “Aminosterol ester compounds,” Zasloff, Kinney, Jones.
U.S. Pat. No. 5,874,597 (1999) for “Certain Aminosterol compounds and pharmaceutical compositions including these compounds,” Jones, Issued Feb. 23, 1999.
U.S. Pat. No. 5,994,336 (1999) for “Method of inhibiting proliferation of cells by administering an aminosterol compound,” Zasloff, Shinnar, Kinney, Rao, Issued Nov. 30, 1999.
U.S. Pat. No. 6,017,906 (2000) for “Polyamine conjugates for treatment of infection,” Mintz, C S et al Intercardia, Inc., Issued Jan. 25, 2000
U.S. Pat. No. 6,143,738 (2000) for “Therapeutic uses for an aminosterol compound,” Zasloff, Issued Nov. 7, 2000
U.S. Pat. No. 6,147,060 (2000) for “Treatment of carcinomas using squalamine in combination with other anti-cancer agents,” Zasloff, Williams, Issued Nov. 14, 2000
U.S. Pat. No. 6,388,108 (2002) for “Aminosterol compounds and uses thereof,” Rao, Feibush, Kinney, Zasloff, Noecker, Issued May 14, 2002.
U.S. Pat. No. 6,596,712 (2003) for “Treatment of carcinomas using squalamine in combination with other anticancer agents or modalities,” Zasloff, Williams, Sokoloff, Issued Jul. 22, 2003.
U.S. Pat. No. 6,962,909 (2005) for “Treatment of neovascularization disorders with squalamine,” Zasloff, Shinnar, Kinney, Jones, Issued Nov. 8, 2005.
U.S. Pat. No. 8,729,058 for “Methods And Compositions For Treating And Preventing Viral Infections,” Zasloff et al., issued May 20, 2014.
Vanhooren, V., Libert, C., “The mouse as a model organism in aging research: usefulness, pitfalls and possibilities,” Ageing Res. Rev., 12: 8-21 (2013).
Verdin et al., “Characterization of a common high-affinity receptor for reovirus serotypes 1 and 3 on endothelial cells,” J. Virol., 63(3): 1318-25 (1989).
White et al., “Therapeutic potential of vasoactive intestinal peptide and its receptors in neurological disorders,” CNSNeurol. Disord. Drug Targets, 9(5): 661-6 (2010).
Williams et al., “Squalamine treatment of human tumors in nu/nu mice enhances platinum-based chemotherapies,” Clin. Cancer Res., 7(3): 724-33 (2001).
WO 96/08270 (1996) for “Method for inhibiting sexually transmitted diseases using Magainin antimicrobials or Squalamine Compounds,” Jacob, Zasloff, Williams, Bedi.
Yeung et al., “Membrane phosphatidylserine regulates surface charge and protein localization,” Science, 319(5860): 210-3 (2008).
Yin et al., “Antiangiogenic treatment delays chondrocyte maturation and bone formation during limb skeletogenesis,” J. Bone Miner. Res., 17(1): 56-65 (2002).
Yun et al., “Identification of Squalamine in the Plasma Membrane of White Blood Cells in the Sea Lamprey,” Petromyzon marinus,” J. LipidRes., 48(12): 2579-2586 (2007).
Zasloff, M., “Antimicrobial peptides of multicellular organisms,” Nature, 415(6870): 389-95 (2002).
Zasloff et al., “A spermine-coupled cholesterol metabolite from the shark with potent appetite suppressant and antidiabetic properties,” Int. J. Obes. Relat. Metab. Disord., 25(5): 689-97 (2001).
Zasloff et al., “Squalamine as a broad-spectrum systemic antiviral agent with therapeutic potential,” Proc. Natl. Acad. Sci. USA, 108(38): 15978-83 (2011).
Zhang et al., “Hypothalamic stem cells control ageing speed partly through exosomal miRNAs,” Nature, 548(7665):52-57 (2017).
Zhang et al., “Hypothalamic programming of systemic ageing involving IKK-β, NF-κB and GnRH,” Nature, 497:211-216 (2013).

1. A pharmaceutical composition formulated for intranasal administration, comprising a low dosage of at least one aminosterol or a pharmaceutically acceptable salt or derivative thereof, wherein the dosage of the aminosterol is subtherapeutic when given orally or by injection.

2. The pharmaceutical composition of claim 1, wherein the dosage of the aminosterol or a pharmaceutically acceptable salt or derivative thereof is:
(a) between about 0.001 to about 6 mg; and/or
(b) selected from the group consisting of about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.25, about 2.3, about 2.4, about 2.5, about 2.6, about 2.7, about 2.75, about 2.8, about 2.9, about 3, about 3.1, about 3.2, about 3.25, about 3.3, about 3.4, about 3.5, about 3.6, about 3.7, about 3.75, about 3.8, about 3.9, about 4, about 4.1, about 4.2, about 4.25, about 4.3, about 4.4, about 4.5, about 4.6, about 4.7, about 4.75, about 4.8, about 4.9, about 5, about 5.1, about 5.2, about 5.25, about 5.3, about 5.4, about 5.5, about 5.6, about 5.7, about 5.75, about 5.8, about 5.9, and about 6 mg; and/or
(c) from about 0.001 to 4 mg/kg; and/or
(d) about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.25, about 1.3, about 1.4, about 1.5, about 1.6, about 1.7, about 1.75, about 1.8, about 1.9, about 2, about 2.1, about 2.2, about 2.25, about 2.3, about 2.4, about 2.5, about 2.6, about 2.7, about 2.75, about 2.8, about 2.9, about 3, about 3.1, about 3.2, about 3.25, about 3.3, about 3.4, about 3.5, about 3.6, about 3.7, about 3.75, about 3.8, about 3.9, about 4 mg/kg.

3. The pharmaceutical composition of claim 1, comprising a pharmaceutically acceptable grade of at least one aminosterol or a pharmaceutically acceptable salt or derivative thereof.

4. The pharmaceutical composition of claim 1, wherein the aminosterol is:
(a) isolated from the liver of Squalus acanthias; and/or
(b) a squalamine isomer; and/or
(c) squalamine; and/or
(d) the phosphate salt of squalamine; and/or
(e) comprises a sterol nucleus and a polyamine attached at any position on the sterol, such that the molecule exhibits a net charge of at least +1; and/or
(f) comprises a bile acid nucleus and a polyamine, attached at any position on the bile acid, such that the molecule exhibits a net charge of at least +1, and optionally wherein the polyamine contributes to the net charge; and/or
(g) a derivative modified to include one or more of the following:
(i) substitutions of the sulfate by a sulfonate, phosphate, carboxylate, or other anionic moiety chosen to circumvent metabolic removal of the sulfate moiety and oxidation of the cholesterol side chain;
(ii) replacement of a hydroxyl group by a non-metabolizable polar substituent, such as a fluorine atom, to prevent its metabolic oxidation or conjugation; and
(iii) substitution of one or more ring hydrogen atoms to prevent oxidative or reductive metabolism of the steroid ring system; and/or
(h) a derivative of squalamine modified through medical chemistry to improve bio-distribution, ease of administration, metabolic stability, or any combination thereof;
(i) aminosterol 1436; and/or
(j) an isomer of aminosterol 1436; and/or
(k) the phosphate salt of aminosterol 1436; and/or
(l) a pharmaceutically acceptable salt of the aminosterol, wherein the salt has low mucosal irritation; and/or
(m) a synthetic aminosterol; and/or
(n) a free base of an aminosterol.

5. The pharmaceutical composition of claim 1, further comprising:
(a) an aqueous carrier;
(b) a buffer;
(c) a sugar, which is optionally lactose; and/or
(d) a polyol compound, which is optionally glycerin.

6. The pharmaceutical composition of claim 5, wherein the composition comprises an aqueous carrier and glycerin at about a 2:1 ratio.

7. A method of treating a subject in need, comprising administering a pharmaceutical composition formulated for intranasal administration, comprising a low dosage of at least one aminosterol or a pharmaceutically acceptable salt or derivative thereof, wherein the dosage of the aminosterol is subtherapeutic when given orally or by injection.

8. (canceled)

9. The method of claim 7, wherein:
(a) the subject is a human; and/or
(b) the subject is an infant, a toddler, a school-aged child, a teenager, a young adult, an adult, or an elderly patient.

10. The method of claim 7, wherein:
(a) the aminosterol is administered in combination with at least one additional active agent to achieve either an additive or synergistic effect; and/or
(b) the aminosterol is administered in combination with at least one additional active agent to achieve either an additive or synergistic effect and wherein the additional active agent is administered via a method selected from the group consisting of concomitantly, as an admixture, separately and simultaneously or concurrently, and separately and sequentially.

11. The method of claim 7, wherein the additional active agent is an aminosterol which is delivered orally.

12. The method of claim 11, wherein the aminosterol administered intranasally is aminosterol 1436 or a salt or derivative thereof, and the aminosterol administered orally is squalamine or a salt or derivative thereof.

13. The method of claim 7, wherein the subject is at risk for developing, or is suffering from, neurodegeneration, and the method results in treating, preventing, and/or delaying the progression and/or onset of neurodegeneration in the subject.

14. The method of claim 13, wherein:
(a) the neurodegeneration is age-related; and/or
(b) the neurodegeneration is correlated with age-related dementia; and/or
(c) the neurodegeneration is correlated with a neurodisease; and/or
(d) the neurodegeneration is correlated with one or more conditions or diseases selected from the group consisting of Alzheimer's disease, Parkinson's disease, Lewy Body dementia, frontotemporal dementia, supranuclear palsy, multi-system atrophy, Parkinsonism, amyotrophic lateral sclerosis (ALS), Huntington's Disease, schizophrenia, Friedreich's ataxia, Multiple sclerosis (MS), spinal muscular atrophy, progressive nuclear palsy, degenerative processes associated with aging, dementia of aging, Guadeloupian Parkinsonism, spinocerebellar ataxia, and vascular dementia; and/or
(e) progression or onset of the neurodegeneration is slowed, halted, or reversed over a defined time period following administration of the pharmaceutical composition, as measured by a medically-recognized technique; and/or
(f) the neurodegeneration is positively impacted by administration of the pharmaceutical composition, and optionally wherein the positive impact and/or progression of neurodegeneration is measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; and/or
(g) the progression or onset of neurodegeneration is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(i) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

15. The method of claim 7, wherein the subject is at risk of developing, or suffers from, a sleep disorder or sleep disturbance, and optionally wherein:
(a) the method or composition results in a positive change in the sleeping pattern of the subject, and optionally wherein the positive change is defined as:
(i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(b) as a result of the method or composition the subject obtains the total number of hours of sleep recommended by a medical authority for the age group of the subject.

16. The method of claim 15, wherein:
(a) administration of the composition decreases the occurrence of at least one symptom of the sleep disorder or disturbance; and/or
(b) the sleep disorder comprises a loss of diurnal rhythm (Circadian rhythm), and optionally wherein the loss of diurnal rhythm is caused by:
(i) dysfunction of the suprachiasmatic nucleus, and optionally wherein the aminosterol reverses the dysfunction of the suprachiasmatic nucleus, restores the diurnal rhythm, and treats the sleep disorder;
(ii) dysfunction of the enteric nervous system, and optionally wherein the aminosterol reverses the dysfunction of the enteric nervous system, restores the diurnal rhythm, and treats the sleep disorder;
(iii) dysfunction of the olfactory nervous system, and optionally wherein the aminosterol reverses the dysfunction of the olfactory system, restores the diurnal rhythm, and treats the sleep disorder;
(iv) visual loss, and optionally wherein the aminosterol reverses the dysfunction of the circadian rhythm caused by visual loss;
(v) jet lag, and optionally wherein the aminosterol reverses the dysfunction of the circadian rhythm caused by jet lag; and/or
(vi) night-shift work, and optionally wherein the aminosterol reverses the dysfunction of the circadian rhythm caused by night-shift work.

17. The method of claim 15, wherein:
(a) the sleep disorder comprises a delay in sleep onset, sleep fragmentation, REM-behavior disorder, sleep-disordered breathing including snoring and apnea, day-time sleepiness, micro-sleep episodes, narcolepsy, hallucinations, or any combination thereof, and optionally wherein the REM-behavior disorder comprises vivid dreams, nightmares, and acting out the dreams by speaking or screaming, or fidgeting or thrashing of arms or legs during sleep; and/or
(b) the sleep disorder is associated with a neurodegenerative disorder; and/or
(c) treating the sleep disorder prevents or delays the onset or progression of a neurodegenerative disorder; and/or
(d) the sleep disorder is associated with a neurodegenerative disorder and optionally wherein the neurodegenerative disorder is selected from the group consisting of Alzheimer's disease, Parkinson's disease, Lewy Body dementia, frontotemporal dementia, supranuclear palsy, multi-system atrophy, Parkinsonism, amyotrophic lateral sclerosis (ALS), Huntington's Disease, schizophrenia, Friedreich's ataxia, Multiple sclerosis (MS), spinal muscular atrophy, progressive nuclear palsy, degenerative processes associated with aging, dementia of aging, Guadeloupian Parkinsonism, spinocerebellar ataxia, and vascular dementia.

18. The method of claim 7, wherein the subject suffers anosmia or from hyposmia, and the method result in either complete or partial restoration of the subject's sense of smell, and optionally wherein:
(a) the method or pharmaceutical composition results in improving the subject's sense of smell by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(b) the subject:
(i) has experienced head trauma; and/or
(ii) is at risk of developing Parkinson's disease; and/or
(iii) is at risk of developing a neurodisease.

19. The method of claim 7, wherein the subject suffers from, is or at risk of developing, hallucinations, and optionally wherein:
(a) the hallucination comprises a visual, auditory, tactile, gustatory or olfactory hallucination; and/or
(b) the hallucination is the result of:
(i) a neurodegenerative disorder, and optionally wherein the neurodegenerative disorder is selected from the group consisting of Alzheimer's disease, Parkinson's disease, Lewy Body dementia, frontotemporal dementia, supranuclear palsy, multi-system atrophy, Parkinsonism, amyotrophic lateral sclerosis (ALS), Huntington's Disease, schizophrenia, Friedreich's ataxia, Multiple sclerosis (MS), spinal muscular atrophy, progressive nuclear palsy, degenerative processes associated with aging, dementia of aging, Guadeloupian Parkinsonism, spinocerebellar ataxia, and vascular dementia;
(ii) a psychiatric disorder, and optionally wherein the psychiatric disorder is selected from the group consisting of Bipolar disorder, Borderline personality disorder, Depression (mixed), Dissociative identity disorder, Generalized anxiety disorder, Major depression, Obsessive compulsive disorder, Post-traumatic stress disorder, Psychosis (NOS), Schizoaffective disorder, and Schizophrenia;
(iii) a neurological disorder;
(iv) a brain tumor;
(v) a sensory loss, and optionally wherein the sensory loss is visual, auditory, gustatory, tactile, or olfactory; and/or
(vi) dysfunction of the enteric nervous system; and/or
(c) the method or pharmaceutical composition results in a decreased number or severity of hallucinations of the subject, and optionally wherein the decrease in number or severity in hallucinations is defined as a reduction in occurrences or severity of hallucinations selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(d) the method or pharmaceutical composition results in the subject being hallucination-free.

20. The method of claim 7, wherein the subject suffers from, is or at risk of developing, depression, and optionally wherein:
(a) the method or pharmaceutical composition results in improvement in a subject's depression, as measured by one or more clinically-recognized depression rating scale, and optionally wherein:
(i) the improvement is in one or more depression characteristics selected from the group consisting of mood, behavior, bodily functions such as eating, sleeping, energy, and sexual activity, and/or episodes of sadness or apathy; and/or
(ii) the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%; and/or
(b) administration of the intranasal aminosterol composition triggers neurogenesis, which functions to combat depression.

21. The method of claim 7, wherein the subject suffers from, is or at risk of developing, autism, and optionally wherein:
(a) the method or pharmaceutical composition results in improvement in one or more of the subject's autism characteristics or behaviors, as measured by a clinically-recognized rating scale; and/or the method results in improvement in one or more autism characteristics or behaviors selected from the group consisting of social skills, repetitive behaviors, speech, nonverbal communication, sensory sensitivity, behavior, social interaction, and communication skills, as measured using a clinically-recognized scale, and optionally wherein the improvement a subject experiences following treatment in one or more autism characteristics or behaviors is about 5, about 10, about 15, about 20, about 25, about 30, about 35, about 40, about 45, about 50, about 55, about 60, about 65, about 70, about 75, about 80, about 85, about 90, about 95 or about 100%; and/or
(b) administration of the intranasal aminosterol composition triggers neurogenesis, which functions to combat one or more autism characteristics.

22. The method of claim 7, wherein the subject suffers from, is or at risk of developing, schizophrenia, and optionally wherein:
(a) the method or pharmaceutical composition results in improvement in one or more schizophrenia characteristics or behaviors, as measured using a clinically recognized rating scale, and optionally wherein the improvement a subject experiences in one or more schizophrenia characteristics or behaviors following treatment is about 5, about 10, about 15, about 20, about 25, about 30, about 35, about 40, about 45, about 50, about 55, about 60, about 65, about 70, about 75, about 80, about 85, about 90, about 95 or about 100%; and/or
(b) the schizophrenia characteristics or behaviors are selected from the group consisting of unclear or confusing thinking, reduced social engagement, reduced emotional expression, abnormal social behavior, failure to understand reality, lack of motivation, and hearing voices that others do not hear, as measured using a clinically-recognized scale; and/or
(c) administration of the intranasal aminosterol composition triggers neurogenesis, which functions to combat one or more schizophrenia characteristics.

23. The method of claim 7, wherein the subject suffers from, is or at risk of developing, an inflammatory disease or condition caused by excessive expression or concentration of alpha synuclein in the subject, and optionally wherein:
(a) the method or pharmaceutical composition results in a decrease in intensity of inflammation, blood levels of inflammatory markers, inflammatory markers in tissue, number of inflammatory cells in tissue, or any combination thereof, as compared to a control or as compared to the qualitative or quantitative amount from the same patient or subject prior to treatment; and/or
(b) the method or pharmaceutical composition results in a decrease in concentration of alpha synuclein in the subject, and optionally wherein the decrease in alpha-synuclein concentration in is measured qualitatively, quantitatively, or semi-quantitatively by one or more methods selected from the group consisting of:
(i) first determining the concentration of alpha-synuclein in a tissue sample from the subject prior to treatment, followed by: (i) after treatment determining the alpha-synuclein concentration in the same tissue type from the same subject; or (ii) after treatment comparing the alpha-synuclein concentration in the same tissue type to a control;
(ii) measuring the intensity of inflammation over time;
(iii) measuring the amount of inflammatory markers over time;
(iv) measuring the amount of inflammatory markers in blood, plasma, or tissue over time, either qualitatively or quantitatively;
(v) measuring the amount of one or more inflammatory marker cytokines in blood, plasma, or tissue over time, either qualitatively or quantitatively;
(vi) measuring the amount of one or more plasma markers of inflammation such as TNF, IL-8, or CRP in blood, plasma, or tissue over time, either qualitatively or quantitatively; and
(vii) measuring the amount of inflammatory cells in blood, plasma, or tissue over time, either qualitatively or quantitatively; and/or
(c) the method or pharmaceutical composition results in a decrease in concentration of alpha synuclein in the subject and wherein the decrease is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(d) the method or pharmaceutical composition is applied to a patient population susceptible to excessive expression of alpha-synuclein, resulting in an excessive or high concentration of alpha-synuclein.
